(19) United States
(12) Patent Application Publication
(10) Pub. No.: US 2013/0183737 A1
(43) Pub. Date: Jul. 18, 2013

(54) NOVEL BIOMARKERS OF LIVER CANCER

(71) Applicant: Jurgen Borlak, Lehrte/Immensen (DE)

(72) Inventor: Jurgen Borlak, Lehrte/Immensen (DE)

(73) Assignee: MODPRO AB, Uppsala (SE)

(21) Appl. No.: 13/805,475

(22) PCT Filed: Jun. 23, 2011
(86) PCT No.: PCT/EP2011/003185

§ 371 Date: Apr. 3, 2013

(30) Foreign Application Priority Data
Jun. 24, 2010 (EP) 10006565.6

(51) Int. Cl. G01N 33/68

(52) U.S. Cl. CPC G01N 33/6893
USPC 435/190; 435/227; 435/183; 435/200; 435/232; 435/197; 435/196; 530/350; 530/358; 530/351; 530/359; 530/382

(57) ABSTRACT

The invention is based on the surprising finding that proteins regulated by excessive EGFR signalling in the liver may be used as biomarkers in the diagnosis, prognosis and/or monitoring of treatment of diseases, including liver cell dysplasia or hepatocellular carcinoma (HCC), wherein the protein is selected from a first group consisting of Arginase type II, 4931406C07Rik (Ester hydrolase C11orf54 homolog), Akr1c12 protein, Alanyl-tRNA synthetase, Aldo-keto reductase family 1 member C14, Aldo-keto reductase family 1 member C6, Aldolase 3, Alpha glucosidase 2, Beta 5-tubulin, Cai protein (Pdia4), cDNA sequence BC021917 (dihydroxyacetone kinase 2 homolog), Farnesyl diphosphate synthetase, Fatty acid binding protein 5 epidermal, Inosine triphosphatase, Interleukin 25, Kininogen 1, LIM and SH3 protein 1, Major vault protein, Nucb1 protein, Poly(rC) binding protein 2; heterogeneous nuclear ribonucleoprotein X, Psmd11 protein, RIKEN cDNA 2410004H02, Rps12 protein, Sars1 protein, Sorcin, T43799 proteasome protein p45/SUG [imported], Uap1 l1 protein, v-crk sarcoma virus CT10 oncogene homolog, and 170 kDa glucose regulated protein GRP170 precursor, or from a second group consisting of 2-hydroxyphytanoyl-CoA lyase, Branched chain ketoacid dehydrogenase E1 alpha polypeptide; Butyryl Coenzyme A synthetase 1, Dhdh protein, Diacetyl/L-xylulose reductase, Dmgdh protein (Dimethylglycine dehydrogenase, mitochondrial), Enoyl coenzyme A hydratase 1 peroxisomal, Hypothetical protein LOC68347, Lysophospholipase 1, Mitochondrial acyl-CoA thioesterase 1, PREDICTED: agmatine ureohydrolase (agmatinase), RIKEN cDNA 1810013B01 (abhydrolase domain containing 14b), and Serpinb1a protein. Based on this finding novel biomarkers and molecules binding to said biomarkers, compositions and a kit, as well as methods for the diagnosis, prognosis and/or monitoring of treatment of dysplasia and cancer patients, in particular of liver cell dysplasia and hepatocellular carcinoma (HCC) patients are provided according to the invention.

The invention is directed to biomarkers of dysplasia and cancer and the use thereof, in particular in the diagnosis, prognosis and/or monitoring of treatment of liver cell dysplasia or hepatocellular carcinoma (HCC). Areas of application are the life sciences: biology, biochemistry, biotechnology, medicine and medical technology.

Hepatocellular carcinoma (HCC) is one of the most common cancers worldwide. It causes approximately one million deaths each year. Discovery of novel HCC markers for early detection of the disease has been the aim of much research.

Disease proteomics is an evolving science and has been applied especially in the fields of cancer research. However, in order to identify regulated proteins a reference map needs to be established. In the case of the liver the inventors and others reported a proteome map and in the efforts of the inventors >150 novel proteins were identified [1]. Studying quantitative differences in protein expression is a delicate but an important part of proteomics and such research requires efficient methods to allow for reproducible and robust data generation. In this regard two-dimensional electrophoresis (2-DE) and MALDI mass spectrometry (MALDI-MS-TOF) are techniques widely applied to proteomic research to probe for disease regulated proteins in cancer such as hepatocellular carcinoma (HCC). So far, most research focused on viral HBV/HCV-associated HCC, but little is know about non-viral HCC. Liver malignancies are common cancers worldwide and are responsible for approximately one million deaths each year, i.e. most HCC patients died quickly because of rapid tumor progression [2]. Early diagnosis of disease will thus improve overall survival. Recent research from the lab of the inventors identified exaggerated EGF signaling as a possible route for hepatocarcinogenesis, but the EGF receptorkinase plays a much wider role in the immortalisation of different cell types [3]. Notably, the epidermal growth factor (EGF) is highly expressed in a number of solid tumors, and its expression correlates well with tumor progression, resistance to chemotherapy, and poor prognosis; consequently, it is an obvious target for the rational design of novel anticancer agents. In the past, the serum proteome of EGF-tumor-bearing mice has been studied to gain more information about disease-regulated proteins in HCC and to search for novel biomarkers at different stages of disease [4]. Next to alpha-fetoprotein (AFP) only a few serological markers are available (e.g. Lens culinaris agglutinin-reactive AFP (AFP-L3) and protein induced by vitamin K absence or antagonist-II (PIVKA-II)), but its diagnostic accuracy is unsatisfactory, because of its low sensitivity and the non-specific correlation between the clinical behavior of HCC and, for instance, AFP blood levels. For this reason, new biomarkers for the diagnosis of HCC are in strong demand by clinicians [5, 6], and more selective tests, such as soluble interleukin-2 receptor levels, are currently being investigated [7].

The aim of the present invention is therefore to provide novel biomarkers and molecules binding to said biomarkers, compositions and a kit, as well as methods for the diagnosis, prognosis and/or monitoring of treatment of dysplasia and cancer patients, in particular of liver cell dysplasia and hepatocellular carcinoma (HCC) patients.

To this end, the implementation of the embodiments and actions as described in the claims provides appropriate means to fulfill these demands in a satisfying manner.

The invention is based on the surprising finding that proteins regulated by excessive EGFR signalling in the liver may be used as biomarkers, in particular as immunohistochemical markers, in the diagnosis, prognosis and/or monitoring of treatment, preferably of/in the early stage, of diseases, including liver cell dysplasia or hepatocellular carcinoma (HCC), in particular of non-viral HCC, wherein the protein is selected from a first group consisting of

Arginase type II, 4931406C07Rik (Ester hydrolase C11orf54 homolog), Akr1c12 protein, Alanyl-tRNA synthetase, Aldo-keto reductase family 1 member C14, Aldo-keto reductase family 1 member C6, Aldolase 3, Alpha glucosidase 2, Beta 5-tubulin, Cai protein (Pdia4), cDNA sequence BC021917 (dihydroxyacetone kinase 2 homolog), Farnesyl diphosphate synthetase, Fatty acid binding protein 5 epidermal, Inosine triphosphatase, Interleukin 25, Kininogen 1, LIM and SH3 protein 1, Major vault protein, Nucb1 protein, Poly(rC) binding protein 2; heterogeneous nuclear ribonucleoprotein X, Psmd11 protein, RIKEN cDNA 2410004H02, Rps12 protein, Sars1 protein, Sorcin, T43799 proteasome protein p45/SUG [imported], Uap1l1 protein, v-crk sarcoma virus CT10 oncogene homolog, and 170 kDa glucose regulated protein GRP170 precursor,
or from a second group consisting of
2-hydroxyphytanoyl-CoA lyase, Branched chain ketoacid dehydrogenase E1 alpha polypeptide, Butyryl Coenzyme A synthetase 1, Dhdh protein, Diacetyl/L-xylulose reductase, Dmgdh protein (Dimethylglycine dehydrogenase, mitochondrial), Enoyl coenzyme A hydratase 1 peroxisomal, Hypothetical protein LOC68347, Lysophospholipase 1, Mitochondrial acyl-CoA thioesterase 1, PREDICTED: agmatine ureohydrolase (agmatinase), RIKEN cDNA 1810013B01 (abhydrolase domain containing 14b), and Serpinb1a protein,
or wherein a combination of said proteins is selected, in particular if said protein(s) is/are combined with at least one other protein of said 41 proteins, and wherein a combination of at least one of the 28 proteins of the first group with at least one of the 13 proteins of the second group is more particular preferred.

In a first aspect, the invention is thus directed to a protein regulated by excessive EGFR signalling in the liver for use as biomarker in the diagnosis, prognosis and/or monitoring of treatment, preferably in the early stage, of diseases, including liver cell dysplasia or hepatocellular carcinoma (HCC), in particular of non-viral HCC, wherein the protein is selected from a first group consisting of

Arginase type II, 4931406C07Rik (Ester hydrolase C11orf54 homolog), Akr1c12 protein, Alanyl-tRNA synthetase, Aldo-keto reductase family 1 member C14, Aldo-keto reductase family 1 member C6, Aldolase 3, Alpha glucosidase 2, Beta 5-tubulin, Cai protein (Pdia4), cDNA sequence BC021917 (dihydroxyacetone kinase 2 homolog), Farnesyl diphosphate synthetase, Fatty acid binding protein 5 epidermal, Inosine triphosphatase, Interleukin 25, Kininogen 1, LIM and SH3 protein 1, Major vault protein, Nucb1 protein, Poly(rC) binding protein 2; heterogeneous nuclear ribonucleoprotein X, Psmd11 protein, RIKEN cDNA 2410004H02, Rps12 protein, Sars1 protein, Sorcin, T43799 proteasome protein p45/SUG [imported], Uap1l1 protein, v-crk sarcoma virus CT10 oncogene homolog, 170 kDa glucose regulated protein GRP170 precursor,
or from a second group consisting of
2-hydroxyphytanoyl-CoA lyase, Branched chain ketoacid dehydrogenase E1 alpha polypeptide, Butyryl Coenzyme A synthetase 1, Dhdh protein, Diacetyl/L-xylulose reductase, Dmgdh protein (Dimethylglycine dehydrogenase, mitochondrial), Enoyl coenzyme A hydratase 1 peroxisomal, Hypothetical protein LOC68347, Lysophospholipase 1, Mitochondrial acyl-CoA thioesterase 1, PREDICTED: agmatine ureohydrolase (agmatinase), RIKEN cDNA 1810013B01 (abhydrolase domain containing 14b), Serpinb1a protein.

The term hepatocellular carcinoma (HCC) according to the invention is in particular directed to all primary malignancies of the liver, including malignancies induced by cirrhosis or by viral hepatide, in particular hepaptitis B or C, infections, more particular the term hepatocellular carcinoma (HCC) is directed to non-viral HCC, preferably HCC caused by excessive EGFR signalling in the liver.

The proteins according to the invention concern gene products of mammalia, preferably gene products of the genome of mus musculus or homo sapiens, in particular the respective gene products of homo sapiens are preferred, or, respectively, sequence fragments of said gene products as described herein.

Within the context of the invention the term EGFR is directed to mammalian epidermal growth factor receptor (EGFR, also named ErbB-1), preferably murine or human EGFR, in particular human EGFR, and also to oligomers composed of proteins of the epidermal growth factor receptor family (EGFR, HER2, HER3, and HERF2) wherein the oligomers comprise EGFR.

According to the invention the term “regulated by excessive EGFR signalling in the liver” as common technical feature of the 41 biomarker proteins mentioned herein, said term is particularly directed to the regulation of said proteins by exaggerated EGFR tyrosine kinase activity in the liver, in particular induced by overexpression of EGFR in the liver and/or induced by the activation of EGFR by a growth factor selected from the group consisting of EGF, amphiregulin, epiregulin, TGF-α, HB-EGF, betacellulin, epigen, more particular selected from the group consisting of EGF, amphiregulin, epiregulin.

The term “dysplasia” according to the invention is directed to low grade and/or high grade dysplasia, wherein “low grade dysplasia” is particularly directed to a lesion having minimal aberration inside the cell, and “high grade dysplasia” also comprises mild or medium dysplasia. The term “liver cell dysplasia” according to the invention is in particular directed to premalignant lesions of HCC, as described for example by Kojiro M. J Hepatobiliary Pancreat Sum. 2000; 7(6):535-41.

In one aspect, the invention is directed to a protein for use as biomarker in the diagnosis, prognosis and/or treatment monitoring of dysplasia or cancer, in particular bladder, breast, cervical, colorectal, endometrial, gastric, head and neck, ovarian and oesophageal dysplasia or cancer, wherein the protein is selected from a first group consisting of

4931406C07Rik (Ester hydrolase C11orf54 homolog), Akr1c12 protein, Alanyl-tRNA synthetase, Aldo-keto reductase family 1 member C14, Aldo-keto reductase family 1 member C6, Alpha glucosidase 2, Inosine triphosphatase, Interleukin 25, Poly(rC) binding protein 2; heterogeneous nuclear ribonucleoprotein X, RIKEN cDNA 2410004H02, T43799 proteasome protein p45/SUG [imported], Uap1l1 protein, 170 kDa glucose regulated protein GRP170 precursor,
or from a second group consisting of
2-hydroxyphytanoyl-CoA lyase, Branched chain ketoacid dehydrogenase E1 alpha polypeptide, Butyryl Coenzyme A synthetase 1, Dmgdh protein (Dimethylglycine dehydrogenase, mitochondrial), Hypothetical protein LOC68347, Lysophospholipase 1, Mitochondrial acyl-CoA thioesterase 1, PREDICTED: agmatine ureohydrolase (agmatinase), RIKEN cDNA 1810013B01 (abhydrolase domain containing 14b), Serpinb1a protein.

In another aspect, the invention is directed to a protein regulated by excessive EGFR signalling in the liver for use as serum marker in the diagnosis, prognosis and/or treatment monitoring of liver cell dysplasia or hepatocellular carcinoma (HCC) wherein the protein is selected from a first group consisting of

Apolipoprotein A1, Apolipoprotein E, Carboxylesterase precursor, Fibrinogen-alpha polypeptide, Fibrinogen-beta polypeptide, Fibrinogen-gamma polypeptide, Pzp (A2mg protein), Serum amyloid P-component
or from a second group consisting of
Major urinary protein 1.

The term “serum marker” as used herein is in particular understood as a specific indicator found in a blood test, including tests on plasma or serum, that identifies a disease.

The term “serum” according to the invention is in particular directed to blood, plasma, and serum, more particular to blood plasma and serum, wherein blood serum is particularly preferred within the context of the invention.

The term “tissue” according to the invention is directed to the cellular organizational level intermediate between cells and the complete organism, in particular to an ensemble of cells from the same origin that together carry out a specific biological function, and wherein the tissue may be either part of an animal or human body or wherein preferably the tissue has been removed from an animal or human body.

Accordingly, the proteins according to the invention are preferably used as immunohistochemical markers, such as for a immunohistochemical staining, or as blood plasma markers or particularly as blood serum markers.

The invention is further directed to molecules specifically binding to the protein biomarkers mentioned herein or to mRNA coding for said proteins, and wherein said molecules are selected from the group consisting of antibodies and siRNA. The terms “specifically binding” or “specific for” as mentioned herein is particularly related to an association of the biomarker or mRNA with the binding molecule being established via an association constant Ka>1000 M⁻¹.

Thus, the invention is also directed to an antibody specific for a protein regulated by excessive EGFR signalling in the liver for use in the diagnosis, prognosis and/or treatment monitoring of liver cell dysplasia or HCC.protein, wherein the protein is selected from a first group consisting of Arginase type II, 4931406C07Rik (Ester hydrolase C11orf54 homolog), Akr1c12 protein, Alanyl-tRNA synthetase, Aldo-keto reductase family 1 member C14, Aldo-keto reductase family 1 member C6, Aldolase 3, Alpha glucosidase 2, Beta 5-tubulin, Cai protein (Pdia4), cDNA sequence BC021917 (dihydroxyacetone kinase 2 homolog), Farnesyl diphosphate synthetase, Fatty acid binding protein 5 epidermal, Inosine triphosphatase, Interleukin 25, Kininogen 1, LIM and SH3 protein 1, Major vault protein, Nucb1 protein, Poly(rC) binding protein 2; heterogeneous nuclear ribonucleoprotein X, Psmd11 protein, RIKEN cDNA 2410004H02, Rps12 protein, Sars1 protein, Sorcin, T43799 proteasome protein p45/SUG [imported], Uap1l1 protein, v-crk sarcoma virus CT10 oncogene homolog, 170 kDa glucose regulated protein GRP170 precursor, or from a second group consisting of

2-hydroxyphytanoyl-CoA lyase, Branched chain ketoacid dehydrogenase E1 alpha polypeptide, Butyryl Coenzyme A synthetase 1, Dhdh protein, Diacetyl/L-xylulose reductase, Dmgdh protein (Dimethylglycine dehydrogenase, mitochondrial), Enoyl coenzyme A hydratase 1 peroxisomal, Hypothetical protein LOC68347, Lysophospholipase 1, Mitochondrial acyl-CoA thioesterase 1, PREDICTED: agmatine ureohydrolase (agmatinase), RIKEN cDNA 1810013B01 (abhydrolase domain containing 14b), Serpinb1a protein,
and wherein the use of an antibody directed against a protein selected from said first group is particularly preferred.

Further, the invention is thus directed to an antibody specific for a protein for use in the diagnosis, prognosis and/or treatment monitoring of dysplasia or cancer, in particular bladder, breast, cervical, colorectal, endometrial, gastric, head and neck, ovarian and oesophageal dysplasia or cancer, wherein the protein is selected from a first group consisting of

4931406C07Rik (Ester hydrolase C11orf54 homolog), Akr1c12 protein, Alanyl-tRNA synthetase, Aldo-keto reductase family 1 member C14, Aldo-keto reductase family 1 member C6, Alpha glucosidase 2, Inosine triphosphatase, Interleukin 25, Poly(rC) binding protein 2; heterogeneous nuclear ribonucleoprotein X, RIKEN cDNA 2410004H02, T43799 proteasome protein p45/SUG [imported], Uap1l1 protein, 170 kDa glucose regulated protein GRP170 precursor,

or from a second group consisting of

2-hydroxyphytanoyl-CoA lyase, Branched chain ketoacid dehydrogenase E1 alpha polypeptide, Butyryl Coenzyme A synthetase 1, Dmgdh protein (Dimethylglycine dehydrogenase, mitochondrial), Hypothetical protein LOC68347, Lysophospholipase 1, Mitochondrial acyl-CoA thioesterase 1, PREDICTED: agmatine ureohydrolase (agmatinase), RIKEN cDNA 1810013B01 (abhydrolase domain containing 14b), Serpinb1a protein,
and wherein the use of an antibody directed against a protein selected from said first group is particularly preferred.

Moreover, the invention is thus directed to an antibody for serum profiling in the diagnosis, prognosis and/or treatment monitoring of liver cell dysplasia or HCC, wherein the antibody is specific for a protein selected from a first group consisting of Apolipoprotein A1, Apolipoprotein E, Carboxylesterase precursor, Fibrinogen-alpha polypeptide, Fibrinogen-beta polypeptide, Fibrinogen-gamma polypeptide, Pzp (A2mg protein), Serum amyloid P-component

or selected from a second group consisting of Major urinary protein 1,
and wherein the use of an antibody directed against a protein selected from said first group is particularly preferred.

Within the inventive context, antibodies are understood to include monoclonal antibodies and polyclonal antibodies and antibody fragments (e.g., Fab, and F(ab′)₂) specific for one of said proteins. Polyclonal antibodies against selected antigens may be readily generated by one of ordinary skill in the art from a variety of warm-blooded animals such as horses, cows, goats, rabbits, mice, rats, chicken or preferably of eggs derived from immunized chicken. Monoclonal antibodies may be generated using conventional techniques (see Monoclonal Antibodies, Hybridomas: A New Dimension in Biological Analyses, Plenum Press, Kennett, McKearn, and Bechtol (eds.), 1980, and Antibodies: A Laboratory Manual, Harlow and Lane (eds.), Cold Spring Harbor Laboratory Press, 1988, which are incorporated herein by reference).

The term “serum profiling” according to the invention is in particular directed to the analysis of blood plasma or blood serum for the presence or concentration of the selected protein in said plasma or serum, such as by using a biosensor, performing an ELISA, a Western Blot, a magnetic bead separation/purification, a ZipTip approach, and wherein said procedures, if applicable, may be combined with a mass spectrometry analysis.

The invention is also directed to siRNA, which reduces or preferably inhibits the expression of a protein regulated by excessive EGFR signalling in the liver, for use in the treatment of liver cell dysplasia or HCC, wherein the protein is selected from the group consisiting of Arginase type II, 4931406C07Rik (Ester hydrolase C11orf54 homolog), Akr1c12 protein, Alanyl-tRNA synthetase, Aldo-keto reductase family 1 member C14, Aldo-keto reductase family 1 member C6, Aldolase 3, Alpha glucosidase 2, Beta 5-tubulin, Cai protein (Pdia4), cDNA sequence BC021917 (dihydroxyacetone kinase 2 homolog), Farnesyl diphosphate synthetase, Fatty acid binding protein 5 epidermal, Inosine triphosphatase, Interleukin 25, Kininogen 1, LIM and SH3 protein 1, Major vault protein, Nucb1 protein, Poly(rC) binding protein 2; heterogeneous nuclear ribonucleoprotein X, Psmd11 protein, RIKEN cDNA 2410004H02, Rps12 protein, Sars1 protein, Sorcin, T43799 proteasome protein p45/SUG [imported], Uap1l1 protein, v-crk sarcoma virus CT10 oncogene homolog, 170 kDa glucose regulated protein GRP170 precursor, Apolipoprotein A1, Apolipoprotein E, Carboxylesterase precursor, Fibrinogen-alpha polypeptide, Fibrinogen-beta polypeptide, Fibrinogen-gamma polypeptide, Pzp (A2mg protein), Serum amyloid P-component.

Within this context, the present invention employs siRNA for use in modulating the level of protein presence in the cell, wherein siRNA oligonucleotides specifically hybridize nucleic acids encoding the selected protein and interfere with the expression of the gene coding for said protein.

Preferably, the siRNA comprises double stranded RNA including a sense RNA strand and an antisense RNA strand, wherein the sense RNA strand comprises a subsequence being 15-30, preferably 19, 20, 21, 22, 23, 24, or 25 contiguous RNA nucleotides in length, preferably corresponding to the ORF sequence encoding the selected protein, and wherein said subsequence contains sequences that are complementary and non-complementary to at least a portion of the mRNA coding for the selected protein.

In another aspect, the invention is directed to a nucleotide sequence coding for a protein regulated by excessive EGFR signalling in the liver for use in the treatment of liver cell dysplasia or HCC, wherein the protein is selected from the group consisting of 2-hydroxyphytanoyl-CoA lyase, Branched chain ketoacid dehydrogenase E1 alpha polypeptide, Butyryl Coenzyme A synthetase 1, Dmgdh protein (Dimethylglycine dehydrogenase, mitochondrial), Hypothetical protein LOC68347, Lysophospholipase 1, Mitochondrial acyl-CoA thioesterase 1, PREDICTED: agmatine ureohydrolase (agmatinase), RIKEN cDNA 1810013B01 (abhydrolase domain containing 14b), Serpinb1a protein, Major urinary protein 1.

The nucleotide sequence particularly comprises a nucleic acid being from about 20 base pairs to about 100,000 base pairs in length. Preferably the nucleic acid is from about 50 base pairs to about 50,000 base pairs in length. More preferably the nucleic acid is from about 50 base pairs to about 10,000 base pairs in length. Most preferred is a nucleic acid from about 50 pairs to about 4,000 base pairs in length. The nucleotide sequence can be a gene or gene fragment that encodes the protein, an oligopeptide or a peptide. Preferably, the nucleotide sequence of the present invention may comprise a DNA construct capable of generating the selected protein and may further include an active constitutive or inducible promoter sequence.

In particular the nucleotide composition comprises a nucleotide sequence encoding a polypeptide which has at least 70% identity, preferably at least 80% identity, more preferably at least 90% identity, yet more preferably at least 95% identity, to the amino acid sequence of the selected protein. In this regard, nucleotide sequences coding for polypeptides which have at least 97% identity are highly preferred, whilst those with at least 98-99% identity are more preferred, and those with at least 99% identity are most preferred. In particular, it is preferred if the nucleotide sequence encodes a polypeptide with 100% identity to the entire amino acid sequence of the selected protein.

In particular, the nucleotide composition comprises a DNA sequence that has at least 70% identity, preferably at least 80% identity, more preferably at least 90% identity, yet more preferably at least 95% identity, to the ORF (or coding sequence, respectively) of the selected protein over the entire coding region. In this regard, nucleoetide sequences which have at least 97% identity are highly preferred, whilst those with at least 98-99% identity are more highly preferred, and those with at least 99% identity are most highly preferred. In particular, it is preferred if the nucleotide sequence encodes a DNA sequence that has 100% identity to the entire ORF of the selected protein over the entire coding region.

In another aspect the nucleotide sequence composition may further comprise an enhancer element and/or a promoter located 5′ to and controlling the expression of said therapeutic nucleotide sequence or gene. The promoter is a DNA segment that contains a DNA sequence that controls the expression of a gene located 3′ or downstream of the promoter. The promoter is the DNA sequence to which RNA polymerase specifically binds and initiates RNA synthesis (transcription) of that gene, typically located 3′ of the promoter.

Further, within the context of the present invention an antisense composition is provided for use in the treatment of liver cell dysplasia or HCC, wherein the antisense composition comprises a nucleotide sequence complementary to a coding sequence of a protein regulated by excessive EGFR signalling in the liver, wherein the protein is selected from the group consisting of Arginase type II, 4931406C07Rik (Ester hydrolase C11orf54 homolog), Akr1c12 protein, Alanyl-tRNA synthetase, Aldo-keto reductase family 1 member C14, Aldo-keto reductase family 1 member C6, Aldolase 3, Alpha glucosidase 2, Beta 5-tubulin, Cai protein (Pdia4), cDNA sequence BC021917 (dihydroxyacetone kinase 2 homolog), Farnesyl diphosphate synthetase, Fatty acid binding protein 5 epidermal, Inosine triphosphatase, Interleukin 25, Kininogen 1, LIM and SH3 protein 1, Major vault protein, Nucb1 protein, Poly(rC) binding protein 2; heterogeneous nuclear ribonucleoprotein X, Psmd11 protein, RIKEN cDNA 2410004H02, Rps12 protein, Sars1 protein, Sorcin, T43799 proteasome protein p45/SUG [imported], Uap1l1 protein, v-crk sarcoma virus CT10 oncogene homolog, 170 kDa glucose regulated protein GRP170 precursor, Apolipoprotein A1, Apolipoprotein E, Carboxylesterase precursor, Fibrinogen-alpha polypeptide, Fibrinogen-beta polypeptide, Fibrinogen-gamma polypeptide, Pzp (A2mg protein), Serum amyloid P-component.

Said nucleotide sequences and siRNA according to the invention may be prepared by any standard method for producing a nucleotide sequence or siRNA, such as by recombinant methods, in particular synthetic nucleotide sequences and siRNA is preferred.

Said nucleotide sequences and siRNA are preferably for the use in the treatment of liver cell dysplasia or HCC by administering an amount of said nucleotide sequences and/or siRNA to a subject suffering from or being susceptible to liver cell dysplasia or HCC for decreasing or increasing the expression or biological activity of the targeted protein to a normal level.

The invention thus also relates to a composition comprising a substance that
decreases or inhibits the expression or biological activity of a protein selected from the group consisting of Arginase type II, 4931406C07Rik (Ester hydrolase C11orf54 homolog), Akr1c12 protein, Alanyl-tRNA synthetase, Aldo-keto reductase family 1 member C14, Aldo-keto reductase family 1 member C6, Aldolase 3, Alpha glucosidase 2, Beta 5-tubulin, Cai protein (Pdia4), cDNA sequence BC021917 (dihydroxyacetone kinase 2 homolog), Farnesyl diphosphate synthetase, Fatty acid binding protein 5 epidermal, Inosine triphosphatase, Interleukin 25, Kininogen 1, LIM and SH3 protein 1, Major vault protein, Nucb1 protein, Poly(rC) binding protein 2; heterogeneous nuclear ribonucleoprotein X, Psmd11 protein, RIKEN cDNA 2410004H02, Rps12 protein, Sars1 protein, Sorcin, T43799 proteasome protein p45/SUG [imported], Uap1l1 protein, v-crk sarcoma virus CT10 oncogene homolog, 170 kDa glucose regulated protein GRP170 precursor, Apolipoprotein A1, Apolipoprotein E, Carboxylesterase precursor, Fibrinogen-alpha polypeptide, Fibrinogen-beta polypeptide, Fibrinogen-gamma polypeptide, Pzp (A2mg protein), Serum amyloid P-component, and/or
increases the expression or biological activity of a protein selected from the group consisting of 2-hydroxyphytanoyl-CoA lyase, Branched chain ketoacid dehydrogenase E1 alpha polypeptide, Butyryl Coenzyme A synthetase 1, Dhdh protein, Diacetyl/L-xylulose reductase, Dmgdh protein (Dimethylglycine dehydrogenase, mitochondrial), Enoyl coenzyme A hydratase 1 peroxisomal, Hypothetical protein LOC68347, Lysophospholipase 1, Mitochondrial acyl-CoA thioesterase 1, PREDICTED: agmatine ureohydrolase (agmatinase), RIKEN cDNA 1810013B01 (abhydrolase domain containing 14b), Serpinb1a protein, Major urinary protein 1,
   for the treatment of liver cell dysplasia and HCC, wherein the substance is preferably selected from the group consisting of said nucleotide sequences and siRNA according to the invention.

Preferably, the compositions according to the invention further comprises a pharmaceutically acceptable carrier and/or recipient and/or diluent.

The term “biological activity” within the context of the invention is particularly directed to the interaction of the selected protein with other biomolecules, in particular with proteins, carbohydrates and lipids or with a combination thereof.

The term “subject”, as used herein, is directed to a mammal, in particular to a mouse or a human being having or being susceptible to dysplasia or cancer, preferably liver cell dysplaisa or HCC, more particular to a human dysplasia or cancer patient or a transgenic cancer mouse, including a HCC bearing mouse, such as a patient having liver cell dysplasia or HCC or a transgenic mouse overexpressing Egf, in particular a mouse whose genome comprises a non natural sequence coding for IgEGF, may be.

The invention is also directed to a method of detecting liver cell dysplasia or HCC, in particular non-viral HCC, or of predicting the susceptibility or resistance to liver cell dysplasia or HCC, in particular to non-viral HCC, comprising testing a sample isolated from the liver of a subject for the presence or concentration of a protein selected from a first group consisting of Arginase type II, 4931406C07Rik (Ester hydrolase C11orf54 homolog), Akr1c12 protein, Alanyl-tRNA synthetase, Aldo-keto reductase family 1 member C14, Aldo-keto reductase family 1 member C6, Aldolase 3, Alpha glucosidase 2, Beta 5-tubulin, Cai protein (Pdia4), cDNA sequence BC021917 (dihydroxyacetone kinase 2 homolog), Farnesyl diphosphate synthetase, Fatty acid binding protein 5 epidermal, Inosine triphosphatase, Interleukin 25, Kininogen 1, LIM and SH3 protein 1, Major vault protein, Nucb1 protein, Poly(rC) binding protein 2; heterogeneous nuclear ribonucleoprotein X, Psmd11 protein, RIKEN cDNA 2410004H02, Rps12 protein, Sars1 protein, Sorcin, T43799 proteasome protein p45/SUG [imported], Uap1l1 protein, v-crk sarcoma virus CT10 oncogene homolog, 170 kDa glucose regulated protein GRP170 precursor,

or from a second group consisting of
2-hydroxyphytanoyl-CoA lyase, Branched chain ketoacid dehydrogenase E1 alpha polypeptide, Butyryl Coenzyme A synthetase 1, Dhdh protein, Diacetyl/L-xylulose reductase, Dmgdh protein (Dimethylglycine dehydrogenase, mitochondrial), Enoyl coenzyme A hydratase 1 peroxisomal, Hypothetical protein LOC68347, Lysophospholipase 1, Mitochondrial acyl-CoA thioesterase 1, PREDICTED: agmatine ureohydrolase (agmatinase), RIKEN cDNA 1810013B01 (abhydrolase domain containing 14b), Serpinb1a protein,
or comprising testing a serum sample of a subject for the presence or concentration of a protein from a first group consisting of Apolipoprotein A1, Apolipoprotein E, Carboxylesterase precursor, Fibrinogen-alpha polypeptide, Fibrinogen-beta polypeptide, Fibrinogen-gamma polypeptide, Pzp (A2mg protein), Serum amyloid P-component, or from a second group consisting of Major urinary protein 1,
and wherein, in particular, the sample is tested for the increase of a protein selected from said first group(s) and/or the decrease of a protein selected from said second group(s).

The invention is further directed to a method of detecting the response of a subject to a compound in the treatment of liver cell dysplasia or HCC, or of predicting the responsiveness of subject to a compound in the treatment of liver cell dysplasia or HCC, comprising determining in a sample isolated from the liver of said subject the presence or concentration of a protein regulated by excessive EGFR signalling in the liver, wherein the protein is selected from a first group consisting of Arginase type II, 4931406C07Rik (Ester hydrolase C11orf54 homolog), Akr1c12 protein, Alanyl-tRNA synthetase, Aldo-keto reductase family 1 member C14, Aldo-keto reductase family 1 member C6, Aldolase 3, Alpha glucosidase 2, Beta 5-tubulin, Cai protein (Pdia4), cDNA sequence BC021917 (dihydroxyacetone kinase 2 homolog), Farnesyl diphosphate synthetase, Fatty acid binding protein 5 epidermal, Inosine triphosphatase, Interleukin 25, Kininogen 1, LIM and SH3 protein 1, Major vault protein, Nucb1 protein, Poly(rC) binding protein 2; heterogeneous nuclear ribonucleoprotein X, Psmd11 protein, RIKEN cDNA 2410004H02, Rps12 protein, Sars1 protein, Sorcin, T43799 proteasome protein p45/SUG [imported], Uap1l1 protein, v-crk sarcoma virus CT10 oncogene homolog, 170 kDa glucose regulated protein GRP170 precursor,

or from a second group consisting of
2-hydroxyphytanoyl-CoA lyase, Branched chain ketoacid dehydrogenase E1 alpha polypeptide, Butyryl Coenzyme A synthetase 1, Dhdh protein, Diacetyl/L-xylulose reductase, Dmgdh protein (Dimethylglycine dehydrogenase, mitochondrial), Enoyl coenzyme A hydratase 1 peroxisomal, Hypothetical protein LOC68347, Lysophospholipase 1, Mitochondrial acyl-CoA thioesterase 1, PREDICTED: agmatine ureohydrolase (agmatinase), RIKEN cDNA 1810013B01 (abhydrolase domain containing 14b), Serpinb1a protein,
or comprising determining the presence or concentration of a protein in a serum sample of said subject, wherein the protein is selected from a first group consisting of Apolipoprotein A1, Apolipoprotein E, Carboxylesterase precursor, Fibrinogen-alpha polypeptide, Fibrinogen-beta polypeptide, Fibrinogen-gamma polypeptide, Pzp (A2mg protein), Serum amyloid P-component, or from a second group consisting of Major urinary protein 1,
and wherein the compound is selected from the group of EGF receptor tyrosine kinase activity modulator, siRNA, in particular above-mentioned siRNA, nucleotide sequence, in particular above-mentioned nucleotide sequence, and if applicable said compound is combined with a chemotherapeutic drug,
and wherein the sample is preferably tested for the decrease of the presence or concentration of a protein selected from said first group(s) and/or the increase of the presence or concentration of a protein selected from said second group(s).

The term “EGF receptor tyrosine kinase activity modulator” or “compound modulating EGF receptor tyrosine kinase activity” or “compound to be tested” according to the invention is in particular directed to a compound selected from the group consisting of Sorafenib, Sunitinib, Gefitinib, Erlotinib, anti-EGF antibody, anti-HER1 antibody, anti-HER2 antibody, anti-HER3 antibody, anti-HER4 antibody, Trastuzumab (Herceptin), Cetuximab, Panitumumab, Matuzumab, Nimotuzumab, MDX-447, Pertuzumab.

According to the invention, the term “chemotherapeutic drug” is in particular directed to any antineoplastic chemotherapy drug usable for treating HCC and any chemopreventive drug usable for treating liver cell dysplasia, and wherein the antineoplastic chemotherapy drug is preferably selected from the group consisiting of Taxol, 5-fluorouracil, doxorubicin and vinblastine, or wherein the chemopreventive drug is preferably selected from the group consisiting of Zileuton and Celecoxib.

The invention further concerns a method to screen for and to identify a compound for modulating EGF receptor tyrosine kinase activity in the liver of a subject suffering from or being susceptible to liver cell dysplasia or HCC, comprising the use of a protein biomarker selected from a first group consisting of

Arginase type II, 4931406C07Rik (Ester hydrolase C11orf54 homolog), Akr1c12 protein, Alanyl-tRNA synthetase, Aldo-keto reductase family 1 member C14, Aldo-keto reductase family 1 member C6, Aldolase 3, Alpha glucosidase 2, Beta 5-tubulin, Cai protein (Pdia4), cDNA sequence BC021917 (dihydroxyacetone kinase 2 homolog), Farnesyl diphosphate synthetase, Fatty acid binding protein 5 epidermal, Inosine triphosphatase, Interleukin 25, Kininogen 1, LIM and SH3 protein 1, Major vault protein, Nucb1 protein, Poly(rC) binding protein 2; heterogeneous nuclear ribonucleoprotein X, Psmd11 protein, RIKEN cDNA 2410004H02, Rps12 protein, Sars1 protein, Sorcin, T43799 proteasome protein p45/SUG [imported], Uap1l1 protein, v-crk sarcoma virus CT10 oncogene homolog, 170 kDa glucose regulated protein GRP170 precursor, Apolipoprotein A1, Apolipoprotein E, Carboxylesterase precursor, Fibrinogen-alpha polypeptide, Fibrinogen-beta polypeptide, Fibrinogen-gamma polypeptide, Pzp (A2mg protein), Serum amyloid P-component, or from a second group consisting of
2-hydroxyphytanoyl-CoA lyase, Branched chain ketoacid dehydrogenase E1 alpha polypeptide, Butyryl Coenzyme A synthetase 1, Dhdh protein, Diacetyl/L-xylulose reductase, Dmgdh protein (Dimethylglycine dehydrogenase, mitochondrial), Enoyl coenzyme A hydratase 1 peroxisomal, Hypothetical protein LOC68347, Lysophospholipase 1, Mitochondrial acyl-CoA thioesterase 1, PREDICTED: agmatine ureohydrolase (agmatinase), RIKEN cDNA 1810013B01 (abhydrolase domain containing 14b), Serpinb1a protein, Major urinary protein 1 and/or the use of an antibody specific for one of said proteins.

Thus, the invention is also directed to the use of at least one of said proteins and/or of at least one of said antibodies to screen for and to identify a compound for modulating EGF receptor tyrosine kinase activity in the liver of a subject suffering from or being susceptible to liver cell dysplasia or HCC, in particular by a serum analysis of the subject to which a compound, in particular a (at least putative) EGF receptor tyrosine kinase activity modulator, to be tested has been administered.

In another aspect, the invention is directed to a method of qualifying the EGFR kinase activity in a subject comprising determining in a sample of the liver of a subject suffering from or being susceptible to liver cell dysplasia or HCC at least one protein selected from a first group consisting of

Arginase type II, 4931406C07Rik (Ester hydrolase C11orf54 homolog), Akr1c12 protein, Alanyl-tRNA synthetase, Aldo-keto reductase family 1 member C14, Aldo-keto reductase family 1 member C6, Aldolase 3, Alpha glucosidase 2, Beta 5-tubulin, Cai protein (Pdia4), cDNA sequence BC021917 (dihydroxyacetone kinase 2 homolog), Farnesyl diphosphate synthetase, Fatty acid binding protein 5 epidermal, Inosine triphosphatase, Interleukin 25, Kininogen 1, LIM and SH3 protein 1, Major vault protein, Nucb1 protein, Poly(rC) binding protein 2; heterogeneous nuclear ribonucleoprotein X, Psmd11 protein, RIKEN cDNA 2410004H02, Rps12 protein, Sars1 protein, Sorcin, T43799 proteasome protein p45/SUG [imported], Uap1l1 protein, v-crk sarcoma virus CT10 oncogene homolog, 170 kDa glucose regulated protein GRP170 precursor
and/or at least one protein selected from a second group consisting of
2-hydroxyphytanoyl-CoA lyase, Branched chain ketoacid dehydrogenase E1 alpha polypeptide, Butyryl Coenzyme A synthetase 1, Dhdh protein, Diacetyl/L-xylulose reductase, Dmgdh protein (Dimethylglycine dehydrogenase, mitochondria!), Enoyl coenzyme A hydratase 1 peroxisomal, Hypothetical protein LOC68347, Lysophospholipase 1, Mitochondrial acyl-CoA thioesterase 1, PREDICTED: agmatine ureohydrolase (agmatinase), RIKEN cDNA 1810013B01 (abhydrolase domain containing 14b), Serpinb1a protein,
and/or comprising determining in a serum sample of a subject suffering from or being susceptible to liver cell dysplasia or HCC at least one protein selected from a first group consisting of
Apolipoprotein A1, Apolipoprotein E, Carboxylesterase precursor, Fibrinogen-alpha polypeptide, Fibrinogen-beta polypeptide, Fibrinogen-gamma polypeptide, Pzp (A2mg protein), Serum amyloid P-component
and/or at least one protein selected from a second group consisting of Major urinary protein 1,
wherein the level of the at least one protein of said first group(s) being significantly higher and/or the level of the at least one protein of said second group(s) being significantly lower than the level of said protein(s) in the liver of subjects without cancer associated with increased activity of EGFR is indicative of induced EGFR kinase activity in the subject,
and optionally further comprising the comprising the above-mentioned screening method to identify a compound for modulating the increased EGF kinase activity in the liver of the subject.

The invention also concerns a method, in particular the aforementioned method, for predicting the response of a liver cell dysplasia or HCC patient to the administration of a EGF receptor tyrosine kinase activity modulator, wherein the level of at least one protein selected from said first group(s) of proteins being significantly higher and/or the level of at least one protein selected from said second group(s) of proteins being significantly lower in the liver tissue or serum (or sample sample, respectively) of said patient than the level of said protein(s) in the liver tissue or serum (or sample sample, respectively) of subjects without liver cell dysplasia or HCC associated with increased activity of EGF receptor tyrosine kinase is indicative that the patient will respond therapeutically to a method of treating cancer comprising administering a EGF receptor tyrosine kinase activity modulator.

In a preferred embodiment, the methods of the invention are implemented by performing an immunoassay, such as an enzyme immunoassay (EIA), a radio immunoassay (RIA) or a fluorescence immunoassay (FIA) may be, in particular by using the kit according to the invention and/or by performing an immunohistochemical analysis or a western blot.

Preferably, at least one antibody specific for a protein selected from the group consisting of

Arginase type II, 4931406C07Rik (Ester hydrolase C11orf54 homolog), Akr1c12 protein, Alanyl-tRNA synthetase, Aldo-keto reductase family 1 member C14, Aldo-keto reductase family 1 member C6, Aldolase 3, Alpha glucosidase 2, Beta 5-tubulin, Cai protein (Pdia4), cDNA sequence BC021917 (dihydroxyacetone kinase 2 homolog), Farnesyl diphosphate synthetase, Fatty acid binding protein 5 epidermal, Inosine triphosphatase, Interleukin 25, Kininogen 1, LIM and SH3 protein 1, Major vault protein, Nucb1 protein, Poly(rC) binding protein 2; heterogeneous nuclear ribonucleoprotein X, Psmd11 protein, RIKEN cDNA 2410004H02, Rps12 protein, Sars1 protein, Sorcin, T43799 proteasome protein p45/SUG [imported], Uap1l1 protein, v-crk sarcoma virus CT10 oncogene homolog, 170 kDa glucose regulated protein GRP170 precursor, Apolipoprotein A1, Apolipoprotein E, Carboxylesterase precursor, Fibrinogen-alpha polypeptide, Fibrinogen-beta polypeptide, Fibrinogen-gamma polypeptide, Pzp (A2mg protein), Serum amyloid P-component, 2-hydroxyphytanoyl-CoA lyase, Branched chain ketoacid dehydrogenase E1 alpha polypeptide, Butyryl Coenzyme A synthetase 1, Dhdh protein, Diacetyl/L-xylulose reductase, Dmgdh protein (Dimethylglycine dehydrogenase, mitochondrial), Enoyl coenzyme A hydratase 1 peroxisomal, Hypothetical protein LOC68347, Lysophospholipase 1, Mitochondrial acyl-CoA thioesterase 1, PREDICTED: agmatine ureohydrolase (agmatinase), RIKEN cDNA 1810013B01 (abhydrolase domain containing 14b), Serpinb1a protein, and Major urinary protein 1, is used for the immunoassay and/or reagents effective to detect said biomarker(s) in a serum sample, such as a blocking buffer for reducing unspecific antibody binding or an enzyme substrate for imaging enzyme labelled antibodies may be, is used for the immunoassay.

In particular it is preferred if an immunohistochemical analysis and/or a western blot is performed for determining the presence or concentration of at least one of said proteins, and wherein preferably at least one of said antibodies is used, and/or wherein dysplastic or malignantly transformed cells isolated from liver tissue by laser microdissection are used.

In another preferred embodiment, the method is implemented by performing a peptide mass fingerprinting, in particular by using the kit described herein.

In one embodiment, the methods according to the invention comprise the steps of
adding lysis buffer to a sample, preferably a serum sample or a liver tissue sample, isolated from a subject suffering from or being susceptible to liver cell dysplasia or HCC;
separating the proteins of the lysed serum sample by 2D gel electrophoresis;
excising from the gel at least one 2-D spot containing a differentially regulated protein;
adding digesting buffer, preferably a buffer containing trypsin, to the at least one excised sample;
determining the identity of the protein by analyzing the digested 2-D spot by mass spectrometry.

According to the invention, the identity, or the presence and/or the concentration, respectively, of the proteins Arginase type II, 4931406C07Rik (Ester hydrolase C11orf54 homolog), Akr1c12 protein, Alanyl-tRNA synthetase, Aldo-keto reductase family 1 member C14, Aldo-keto reductase family 1 member C6, Aldolase 3, Alpha glucosidase 2, Beta 5-tubulin, Cai protein (Pdia4), cDNA sequence BC021917 (dihydroxyacetone kinase 2 homolog), Farnesyl diphosphate synthetase, Fatty acid binding protein 5 epidermal, Inosine triphosphatase, Interleukin 25, Kininogen 1, LIM and SH3 protein 1, Major vault protein, Nucb1 protein, Poly(rC) binding protein 2; heterogeneous nuclear ribonucleoprotein X, Psmd11 protein, RIKEN cDNA 2410004H02, Rps12 protein, Sars1 protein, Sorcin, T43799 proteasome protein p45/SUG [imported], Uap1l1 protein, v-crk sarcoma virus CT10 oncogene homolog, 170 kDa glucose regulated protein GRP170 precursor, Apolipoprotein A1, Apolipoprotein E, Carboxylesterase precursor, Fibrinogen-alpha polypeptide, Fibrinogen-beta polypeptide, Fibrinogen-gamma polypeptide, Pzp (A2mg protein), Serum amyloid P-component, 2-hydroxyphytanoyl-CoA lyase, Branched chain ketoacid dehydrogenase E1 alpha polypeptide, Butyryl Coenzyme A synthetase 1, Dhdh protein, Diacetyl/L-xylulose reductase, Dmgdh protein (Dimethylglycine dehydrogenase, mitochondrial), Enoyl coenzyme A hydratase 1 peroxisomal, Hypothetical protein LOC68347, Lysophospholipase 1, Mitochondrial acyl-CoA thioesterase 1, PREDICTED: agmatine ureohydrolase (agmatinase), RIKEN cDNA 1810013B01 (abhydrolase domain containing 14b), Serpinb1a protein, and Major urinary protein 1, may be determined by determining the presence or concentration of fragments, in particular being 7-24 amino acid residues in length, of said proteins, preferably in a tissue or body fluid sample, which may have been further processed, such as by 2-DE, and wherein a protease, preferably trypsin, has been added to said preferably further processed sample.

In particular, a method according to the invention is preferred, wherein peptide mass fingerprinting is performed, preferably based on mass spectrometry with 2D tryptic digested spots selected by recognition and identified by MALDI-TOF, ESI-TOF or ITMS, for determining the presence or concentration of the selected protein, preferably in the serum or tissue.

In another embodiment, a method according to the invention is preferred, wherein the expression of the gene coding for the selected protein is determined by means of a PCR, preferably a RT-PCR and/or a quantitative real time PCR, for determining the presence or concentration of said protein, preferably in the sample of the tissue isolated from of the liver.

The invention is further directed to a kit for the use in qualifying the EGF receptor tyrosine kinase activity in a subject suffering from or being susceptible to cancer or dysplasia, in particular liver cell dysplasia or HCC, preferably for use in a method according to the invention, in particular for predicting or monitoring the response of a liver cell dysplasia or HCC patient to a method of treating cancer comprising administering a EGF receptor tyrosine kinase activity modulator, wherein the kit comprises at least one standard indicative of the level of a protein selected from the group consisting of

Arginase type II, 4931406C07Rik (Ester hydrolase C11orf54 homolog), Akr1c12 protein, Alanyl-tRNA synthetase, Aldo-keto reductase family 1 member C14, Aldo-keto reductase family 1 member C6, Aldolase 3, Alpha glucosidase 2, Beta 5-tubulin, Cai protein (Pdia4), cDNA sequence BC021917 (dihydroxyacetone kinase 2 homolog), Farnesyl diphosphate synthetase, Fatty acid binding protein 5 epidermal, Inosine triphosphatase, Interleukin 25, Kininogen 1, LIM and SH3 protein 1, Major vault protein, Nucb1 protein, Poly(rC) binding protein 2; heterogeneous nuclear ribonucleoprotein X, Psmd11 protein, RIKEN cDNA 2410004H02, Rps12 protein, Sars1 protein, Sorcin, T43799 proteasome protein p45/SUG [imported], Uap1l1 protein, v-crk sarcoma virus CT10 oncogene homolog, 170 kDa glucose regulated protein GRP170 precursor, Apolipoprotein A1, Apolipoprotein E, Carboxylesterase precursor, Fibrinogen-alpha polypeptide, Fibrinogen-beta polypeptide, Fibrinogen-gamma polypeptide, Pzp (A2mg protein), Serum amyloid P-component, 2-hydroxyphytanoyl-CoA lyase, Branched chain ketoacid dehydrogenase E1 alpha polypeptide, Butyryl Coenzyme A synthetase 1, Dhdh protein, Diacetyl/L-xylulose reductase, Dmgdh protein (Dimethylglycine dehydrogenase, mitochondrial), Enoyl coenzyme A hydratase 1 peroxisomal, Hypothetical protein LOC68347, Lysophospholipase 1, Mitochondrial acyl-CoA thioesterase 1, PREDICTED: agmatine ureohydrolase (agmatinase), RIKEN cDNA 1810013B01 (abhydrolase domain containing 14b), Serpinb1a protein, and Major urinary protein 1 in the liver or in a serum sample, of normal individuals or in the liver or serum of individuals having liver cell dysplasia or HCC associated with increased EGF receptor tyrosine kinase activity, and/or at least one preferably synthetic fragment, being 7-24 amino acids in length, and/or at least one antibody specific for said protein(s), and/or at least one primer pair for determining the mRNA coding for the protein, and preferably instructions for the use of the kit.

The invention also concerns the use of at least one biomarker selected from the group consisting of the proteins

Arginase type II, 4931406C07Rik (Ester hydrolase C11orf54 homolog), Akr1c12 protein, Alanyl-tRNA synthetase, Aldo-keto reductase family 1 member C14, Aldo-keto reductase family 1 member C6, Aldolase 3, Alpha glucosidase 2, Beta 5-tubulin, Cai protein (Pdia4), cDNA sequence BC021917 (dihydroxyacetone kinase 2 homolog), Farnesyl diphosphate synthetase, Fatty acid binding protein 5 epidermal, Inosine triphosphatase, Interleukin 25, Kininogen 1, LIM and SH3 protein 1, Major vault protein, Nucb1 protein, Poly(rC) binding protein 2; heterogeneous nuclear ribonucleoprotein X, Psmd11 protein, RIKEN cDNA 2410004H02, Rps12 protein, Sars1 protein, Sorcin, T43799 proteasome protein p45/SUG [imported], Uap1l1 protein, v-crk sarcoma virus CT10 oncogene homolog, 170 kDa glucose regulated protein GRP170 precursor, Apolipoprotein A1, Apolipoprotein E, Carboxylesterase precursor, Fibrinogen-alpha polypeptide, Fibrinogen-beta polypeptide, Fibrinogen-gamma polypeptide, Pzp (A2mg protein), Serum amyloid P-component, 2-hydroxyphytanoyl-CoA lyase, Branched chain ketoacid dehydrogenase E1 alpha polypeptide, Butyryl Coenzyme A synthetase 1, Dhdh protein, Diacetyl/L-xylulose reductase, Dmgdh protein (Dimethylglycine dehydrogenase, mitochondrial), Enoyl coenzyme A hydratase 1 peroxisomal, Hypothetical protein LOC68347, Lysophospholipase 1, Mitochondrial acyl-CoA thioesterase 1, PREDICTED: agmatine ureohydrolase (agmatinase), RIKEN cDNA 1810013B01 (abhydrolase domain containing 14b), Serpinb1a protein, and Major urinary protein 1 and/or of at least one antibody directed against said at least one biomarker, in the diagnosis, prognosis and/or treatment monitoring of cancer or dysplasia, in particular of HCC or liver cell dysplasia.

Preferably, an appropriate amount of the at least one biomarker is used, in particular an amount for manufacturing a reference, more particular for manufacturing a reference comprising a reference level of said at least one biomarker, such as the level of said at least one biomarker in a sample of a normal healthy subject or the level of a said at least one biomarker in a sample of a patient suffering from HCC or having liver cell dysplasia may be.

In particular, at least one of said biomarkers and/or at least one antibody directed against said at least biomarker, is used for monitoring the therapeutic treatment of a patient suffering from HCC or having liver cell dysplasia, in particular the treatment with a chemotherapeutic drug, preferably with an antineoplastic chemotherapy drug, or with a chemopreventive drug.

Further, a composition for diagnosing or treatment monitoring of dysplasia or cancer, in particular of liver cell dysplasia or HCC, associated with increased EGF receptor tyrosine kinase activity in a patient, preferably by an in vitro body fluid analysis, is provided according to the invention, comprising an effective amount of at least one biomarker selected from the group consisting of the proteins Arginase type II, 4931406C07Rik (Ester hydrolase C11orf54 homolog), Akr1c12 protein, Alanyl-tRNA synthetase, Aldo-keto reductase family 1 member C14, Aldo-keto reductase family 1 member C6, Aldolase 3, Alpha glucosidase 2, Beta 5-tubulin, Cai protein (Pdia4), cDNA sequence BC021917 (dihydroxyacetone kinase 2 homolog), Farnesyl diphosphate synthetase, Fatty acid binding protein 5 epidermal, Inosine triphosphatase, Interleukin 25, Kininogen 1, LIM and SH3 protein 1, Major vault protein, Nucb1 protein, Poly(rC) binding protein 2; heterogeneous nuclear ribonucleoprotein X, Psmd11 protein, RIKEN cDNA 2410004H02, Rps12 protein, Sars1 protein, Sorcin, T43799 proteasome protein p45/SUG [imported], Uap1l1 protein, v-crk sarcoma virus CT10 oncogene homolog, 170 kDa glucose regulated protein GRP170 precursor, Apolipoprotein A1, Apolipoprotein E, Carboxylesterase precursor, Fibrinogen-alpha polypeptide, Fibrinogen-beta polypeptide, Fibrinogen-gamma polypeptide, Pzp (A2mg protein), Serum amyloid P-component, 2-hydroxyphytanoyl-CoA lyase, Branched chain ketoacid dehydrogenase E1 alpha polypeptide, Butyryl Coenzyme A synthetase 1, Dhdh protein, Diacetyl/L-xylulose reductase, Dmgdh protein (Dimethylglycine dehydrogenase, mitochondrial), Enoyl coenzyme A hydratase 1 peroxisomal, Hypothetical protein LOC68347, Lysophospholipase 1, Mitochondrial acyl-CoA thioesterase 1, PREDICTED: agmatine ureohydrolase (agmatinase), RIKEN cDNA 1810013B01 (abhydrolase domain containing 14b), Serpinb1a protein, and Major urinary protein 1, or of a preferably synthetic fragment, being 7-24 amino acids in length, of at least one of said proteins, or comprising at least one antibody directed against said at least one biomarker, in particular for use in diagnosing or treatment monitoring of dysplasia or cancer, preferably of liver cell dysplasia or HCC, associated with increased EGF receptor tyrosine kinase activity in a patient.

The term “body fluid” according to the invention is directed to any body fluid of a subject, in particular to intracellular fluid (or cytosol, respectively), blood, plasma, serum or urine, whereas blood serum or plasma is the preferred body fluid within the context of the invention.

Said composition is preferably used for the production of a diagnostic agent, in particular of a diagnostic standard for body fluid analysis, or, more particular, for the production of a diagnostic agent for qualifying the EGF receptor tyrosine kinase activity in a patient suffering or being susceptible to cancer or for classifying a patient suffering from or being susceptible to HCC.

Within this context, said composition is particularly used for the production of a diagnostic agent for predicting or monitoring the response of a cancer patient to a method of treating cancer comprising administering a EGF receptor tyrosine kinase activity modulator, e.g. Sorafenib.

In yet another preferred embodiment, said composition further comprises an effective amount of a protease, in particular of trypsin, thus enabling a further enhancement of the system sensitivity.

Said composition, in particular the protease digest thereof, may be preferably used for producing a vaccine for the immunization of an animal in order to produce polyclonal antibodies specific for the at least one biomarker.

Within the context of the invention, also a method of qualifying the EGF receptor tyrosine kinase activity in a patient suffering or being susceptible to cancer or for classifying a patient suffering from or being susceptible to HCC is provided, comprising determining in a body fluid sample of a subject suffering from or being susceptible to cancer at least one biomarker selected from a first group consisting of the proteins Arginase type II, 4931406C07Rik (Ester hydrolase C11orf54 homolog), Akr1c12 protein, Alanyl-tRNA synthetase, Aldo-keto reductase family 1 member C14, Aldo-keto reductase family 1 member C6, Aldolase 3, Alpha glucosidase 2, Beta 5-tubulin, Cai protein (Pdia4), cDNA sequence BC021917 (dihydroxyacetone kinase 2 homolog), Farnesyl diphosphate synthetase, Fatty acid binding protein 5 epidermal, Inosine triphosphatase, Interleukin 25, Kininogen 1, LIM and SH3 protein 1, Major vault protein, Nucb1 protein, Poly(rC) binding protein 2; heterogeneous nuclear ribonucleoprotein X, Psmd11 protein, RIKEN cDNA 2410004H02, Rps12 protein, Sars1 protein, Sorcin, T43799 proteasome protein p45/SUG [imported], Uap1l1 protein, v-crk sarcoma virus CT10 oncogene homolog, 170 kDa glucose regulated protein GRP170 precursor, Apolipoprotein A1, Apolipoprotein E, Carboxylesterase precursor, Fibrinogen-alpha polypeptide, Fibrinogen-beta polypeptide, Fibrinogen-gamma polypeptide, Pzp (A2mg protein), Serum amyloid P-component, and/or at least one biomarker selected from the second group consisting of the proteins 2-hydroxyphytanoyl-CoA lyase, Branched chain ketoacid dehydrogenase E1 alpha polypeptide, Butyryl Coenzyme A synthetase 1, Dhdh protein, Diacetyl/L-xylulose reductase, Dmgdh protein (Dimethylglycine dehydrogenase, mitochondrial), Enoyl coenzyme A hydratase 1 peroxisomal, Hypothetical protein LOC68347, Lysophospholipase 1, Mitochondrial acyl-CoA thioesterase 1, PREDICTED: agmatine ureohydrolase (agmatinase), RIKEN cDNA 1810013B01 (abhydrolase domain containing 14b), Serpinb1a protein, Major urinary protein 1, wherein the body fluid level of the at least one biomarker of said first group being significantly higher and/or the body fluid level of the at least one biomarker of said second group being significantly lower than the level of said biomarker(s) in the body fluid of subjects without cancer associated with increased activity of EGF receptor tyrosine kinase is indicative of induced EGF receptor tyrosine kinase activity in the subject.

In one embodiment, said method is preferably used for predicting the response of a cancer patient to a method of treating cancer comprising administering a EGF receptor tyrosine kinase activity modulator, wherein the body fluid level of the at least one biomarker of said first group being significantly higher and/or the body fluid level of the at least one biomarker of said second group being significantly lower than the level of said biomarker(s) in the body fluid of subjects without cancer associated with increased activity of EGF receptor tyrosine kinase is indicative that the subject will respond therapeutically to a method of treating cancer comprising administering a EGF receptor tyrosine kinase activity modulator.

In another embodiment, said method is used for monitoring the therapeutically response of a cancer patient to a method of treating cancer comprising administering an EGF receptor tyrosine kinase activity modulator, wherein the body fluid level of the at least one biomarker of said first group before and after the treatment and/or the body fluid level of the at least one biomarker of said second group before and after the treatment is determined, and a significant decrease of said body fluid level(s) of the at least one biomarker of said first group and/or a significant increase of said body fluid level(s) of the at least one biomarker of said second group after the treatment is indicative that the subject therapeutically responds to the administration of the EGF receptor tyrosine kinase activity modulator.

Moreover, a procedure to screen for and to identify drugs against cancer associated with an increased EGF receptor tyrosine kinase activity is provided, comprising determining in a body fluid sample of a transgenic cancer mouse, including a HCC bearing mouse, being treated with a compound to be tested, in particular of a mouse whose genome comprises a non natural sequence coding for IgEGF, at least one biomarker selected from the first group consisting of the proteins Arginase type II, 4931406C07Rik (Ester hydrolase C11orf54 homolog), Akr1c12 protein, Alanyl-tRNA synthetase, Aldo-keto reductase family 1 member C14, Aldo-keto reductase family 1 member C6, Aldolase 3, Alpha glucosidase 2, Beta 5-tubulin, Cai protein (Pdia4), cDNA sequence BC021917 (dihydroxyacetone kinase 2 homolog), Farnesyl diphosphate synthetase, Fatty acid binding protein 5 epidermal, Inosine triphosphatase, Interleukin 25, Kininogen 1, LIM and SH3 protein 1, Major vault protein, Nucb1 protein, Poly(rC) binding protein 2; heterogeneous nuclear ribonucleoprotein X, Psmd11 protein, RIKEN cDNA 2410004H02, Rps12 protein, Sars1 protein, Sorcin, T43799 proteasome protein p45/SUG [imported], Uap1l1 protein, v-crk sarcoma virus CT10 oncogene homolog, 170 kDa glucose regulated protein GRP170 precursor, Apolipoprotein A1, Apolipoprotein E, Carboxylesterase precursor, Fibrinogen-alpha polypeptide, Fibrinogen-beta polypeptide, Fibrinogen-gamma polypeptide, Pzp (A2mg protein), Serum amyloid P-component, and/or at least one biomarker selected from the second group consisting of the proteins 2-hydroxyphytanoyl-CoA lyase, Branched chain ketoacid dehydrogenase E1 alpha polypeptide, Butyryl Coenzyme A synthetase 1, Dhdh protein, Diacetyl/L-xylulose reductase, Dmgdh protein (Dimethylglycine dehydrogenase, mitochondria!), Enoyl coenzyme A hydratase 1 peroxisomal, Hypothetical protein LOC68347, Lysophospholipase 1, Mitochondrial acyl-CoA thioesterase 1, PREDICTED: agmatine ureohydrolase (agmatinase), RIKEN cDNA 1810013B01 (abhydrolase domain containing 14b), Serpinb1a protein, Major urinary protein 1,

wherein the body fluid level of the at least one biomarker of said first group being significantly lower and/or the body fluid level of the at least one biomarker of said second group being significantly higher than the level of said biomarker(s) in the body fluid of an untreated transgenic cancer mouse is indicative of the therapeutic effect of said compound as a EGF receptor tyrosine kinase activity modulator.

For implementing the methods or uses according to the invention, in particular for determining the presence, concentration or expression of a protein, it may be favourable to use one of the following methods—PCR, in vitro translation, RT-PCR, gel electrophoresis, Western Blot, Northern Blot, Southern Blot, ELISA, FACS measurement, chromatographic isolation, UV microscopy, immunohistochemistry, screening of solid phase bound molecules or tissues, mass spectrometra, and/or biosensory investigation—whereby by amplification, isolation, immobilization and/or detection and/or by combinations of thereof a particularly simple conversion of the methods or according to invention is made possible for the examined sample, in particular if furthermore a statistic analysis is accomplished.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

Since the liver produces many blood proteins as pre-proteins which are then delivered into the blood and activated by proteolytic enzymes, serum proteins may be used for disease diagnosis and prognosis. In the present study, two-dimensional electrophoresis (2-DE) and MALDI-MS were employed to identify disease regulated proteins involved in a HCC-transgenic mouse model. A total of 98 proteins showed significant differences in expression levels between non-transgenic healthy controls and HCC-bearing mice. These included 3-phosphoglycerate dehydrogenase, the capping protein alpha 1 subunit, the fibrinogen alpha polypeptide, and the interleukin 1 receptor antagonist protein, which were found exclusively in HCC-bearing mice. On the contrary, arginosuccinate synthetase 1, dimethylglycine dehydrogenase, and glycine N-methyltransferase are examples of proteins identified only in non-transgenic healthy control samples. A total of 42 new proteins differentially expressed have been related for the first time to HCC, among them two aldo-keto reductase family 1 proteins, members C14 and C6, interleukin 25 and the v-crk sarcoma virus CT10 oncogene homolog. Several works on proteomic analysis of HCC-employing cell lines [8-12] and/or animal models [13-15] already reported novel disease-associated biomarkers, but further validation of the results in human clinical samples will be necessary [16]. Finally, serum and liver proteomes of HCC bearing mice were compared and 10 proteins were found to have the same regulation both in sera and liver tissue, thus, providing a direct link between regulated proteins of the tumor and serum proteomes. Obviously, these are highly interesting biomarker candidates for HCC.

### Materials and Methods

Materials:

A UP 200S Sonicator (Dr. Hielscher GmbH, Stuttgart, Germany) was used to homogenize the samples. For the first dimension, immobilized pH-gradient (IPG) strips (17 cm, pH 5-8, 7-10) were purchased from Bio-Rad (Hercules, Calif., USA). The pre-fractionation was carried out with a Rotofor Cell (Bio-Rad). The focusing chamber was a Protean Isoelectric Focusing (IEF) Cell (Bio-Rad). For the second dimension, a Protean plus Dodeca Cell (Bio-Rad) was used. Reagents (tris, urea, thiourea, CHAPS, dithiothreitol, bromophenol blue, glycerin, sodium dodecyl sulfate, glycine, temed, ammoniumperoxodisulfate, ammonium sulfate, ammonium

bicarbonate, colloidal Coomassie Blue, and acrylamide) were purchased from Roth (Karlsruhe, Germany). Iodacetamide was from SERVA (Heidelberg, Germany). Benzonase was purchased from Novagen (Darmstadt, Germany). Ampholytes (Biolyte 3-10) were purchased from Bio-Rad. DeStreak was purchased from Amersham Bioscience (Freiburg, Germany).

Animal Care:

A total of n=12 C57/BI6 male mice (aged 6-8 months), weighing 25-33 g, were housed in Makrolon Type III cages. Drinking water and food (V1124-000, SSNIFF, The Netherlands® were given ad libitum. The temperature and relative humidity were 22±2° C. and 40-70%, respectively. Furthermore, a 12-h day and night cycle was used. For liver explantation, mice were anesthetized with ketamine 10% 100 μL/100 g and xylazine 2% 50 μL/100 g, and after surgical removal the liver was washed until free of blood.

Mouse Liver Sample Preparation:

Approximately 0.1 g of the liver sample was ground in a mortar under liquid nitrogen flow. Then, the samples were processed with 0.5 mL of a buffer containing 40 mM tris base, 7 M urea, 4% CHAPS, 100 mM DTT, and 0.5% (v/v) biolyte 3-10 first (LB2). The suspensions were homogenized by sonication (3×20 s) and after addition of 3 μL of benzonase (endonuclease that degrades DNA and RNA) were incubated at room temperature for 20 min. The samples were then centrifuged at 12,000 g for 20 min. The pellets were washed and sonicated for 5 min with a further 0.5 mL of LB2 and centrifuged at 12,000 g for another 20 min, and the resulting two fractions of supernatant were collected (extract A). Finally, the pellets were redissolved with 0.5 mL of buffer containing 40 mM tris base, 5 M urea, 2 M thiourea, 4% CHAPS, 100 mM DTT, and 0.5% (v/v) biolyte 3-10 (LB3), sonicated, and centrifuged at 12,000 g for 20 min. The pellet was collected, and the supernatant was marked as extract B. From the same animals, a further 0.1-g portion was ground in a mortar, but was now treated with 0.5 mL of LB3. The suspensions were sonicated, incubated with benzonase, and centrifuged. The pellets were then washed with another 0.5 mL of LB3, sonicated, and centrifuged, and the supernatants were collected (extract C). Proteome mapping was done under a variety of conditions, e.g. extraction with lysis buffers 2 and 3. In addition, proteins were separated at two different pH ranges (5-8 and 7-10). A total of 4 experiments were carried out, and duplicate measurements were run for each experiment. The protein concentration of all extracts was determined using the Bradford assay.

Liquid-Phase IEF Pre-Fractionation:

Liquid-phase IEF pre-fractionation was performed in the Rotofor Cell system (Bio-Rad) following the supplier's instructions. Ion exchange membranes were equilibrated overnight in the appropriate electrolyte (anion exchange membranes in NaOH 0.1 M and cation exchange membranes in H3PO4 0.1 M). After four runs ion exchange membranes were always discarded and new membranes were replaced for the other samples. For each run, the electrode chambers were filled with appropriate fresh electrolytes (30 mL). Initially, the cell was filled with pure water and run for 5 min at 5 watts constant power to remove residual ionic contaminants from the membrane core and ion exchange membranes. Approximately 32 mL of LB2 were used to fill the cell. A total of 60 mg of total proteins in approximately 2 mL of LB2 were added to the cell to reach the maximum loadable volume (40 mL). Focusing started at 12 watts constant power. After approximately 4 hours the voltage increased to 3000 V and the wattage decreased to 3 W. The focused proteins were harvested in 20˜1.5 mL fractions, and pH values were checked. Fractions having pH values between 3 and 7.0 were collected and denoted “A-a” (acid). Fractions having pH values >7.0 were collected and denoted “A-b” (basic). Again, the protein concentration was determined for both fractions (A-a and A-b) by the Bradford method. Approximately 30 mg of protein were recovered at the end of the liquid-phase IEF pre-fractionation from an initial 60-mg load. The losses are accounted for by the multi-step pre-fractionation procedure, but are not the result of a precipitate that could not be dissolved in the lysis buffer described herein. After each run the membrane core was cleaned with NaOH 0.1 M overnight and sonicated for 5 min in water before the new focusing.

Two-Dimensional Gel Electrophoresis Isoelectric Focusing (IEF)—First Dimension:

IEF was performed using precast linear IPG strips. The 17-cm IPG strips 7-10 and 5-8 were loaded with 1.5 mg of proteins by active rehydration (12 h, 50 V). Samples destined to be separated by IPG strips 7-10 received an excess of hydroxyethyldisulphide (HED) (DeStreak™) prior to the focusing run. Focusing began at 250 V for 20 min in rapid mode, 10,000 V for 5 h in linear mode, and 10,000 V for 50,000 Vh in rapid mode (for the IPG strips 5-8). IEF for the strips 7-10 was carried out at 250 V for 60 min in rapid mode, 10,000 V for 3 h in linear mode, and 10,000 V for 50,000 Vh in rapid mode. Each sample was analyzed in duplicate. Control and HCC samples were run always at the same time (6 control and 6 HCC samples).

2-DE—Second Dimension:

After IEF, the IPG strips were either stored at −80° C. or transferred to 10 mL of equilibration buffer (6 M urea, 30% w/v glycerin, 2% w/v SDS, 50 mM Tris-HCl pH 8.8) with 2% w/v DTT and 0.5% v/v bromophenol blue solution (0.25% w/v bromophenol blue, 1.5 M Tris-HCl pH 8.8, 0.4% w/v SDS) and incubated for 20 min at room temperature. Strips were removed and incubated in equilibration buffer with 4% w/v iodoacetamide and 0.5% v/v bromophenol blue solution for further 20 min at room temperature. Finally, the strips and 10 μL SDS-PAGE molecular weight standard on filter paper were placed on top of the 20 cm×20.5 cm 12% second-dimension gel (12% v/v acrylamide/bis solution, 375 mM Tris, pH 8.8, 0.1% v/v SDS, 1/2000 TEMED, 0.05% v/v APS). Both were fixed in place with a 0.5% w/v agarose overlay. Gels were run in PROTEAN Plus Dodeca cell from Bio-Rad at 70 V for approximately 14 h, followed by 200 V until the bromophenol blue dye reached the bottom of the gel. The running buffer (25 mM Tris, 0.2 M glycin, 0.1% SDS) was cooled externally to 16° C. Gels/proteins were fixed overnight in 30% ethanol, 2% phosphoric acid, and washed 3×20 min with 2% phosphoric acid. The gels were equilibrated with 15% ammoniumsulfate, 18% ethanol, 2% phosphoric acid for 15 min and finally stained with colloidal Coomassie Blue for 48 h.

Gel Scanning And Image Analysis:

After staining, gels were washed 10 min with pure water and scanned on a Molecular FX Scanner Bio-Rad at 100 μm resolution. Protein spots were imaged first automatically and then manually and analyzed using the PDQues™ software Bio-Rad The normalization was carried out in total density in gel mode according to the manufacturer's recommendation.

Matrix-Assisted Laser Desorption Ionization Mass Spectrometry (MALDI-MS):

Gels were excised using the spot cutter of Bio-Rad and placed into 96-well microtiter plates. Excised gel spots were washed manually with 20 μL of water for 10 min and destained twice, first with 15 μL ammonium bicarbonate 50 mM for 5 min and then with 15 μL 50% ammonium bicarbonate 50 mM—50% acetonitrile for 5 min. Finally, the gel particles were covered by acetonitrile until gel pieces shrunk and left to dry for 10 min. All gels/proteins were digested manually in situ with 4 μL of ammonium bicarbonate 50 mM containing 20 ng rypsin (Sequencing Grade Modified Trypsin Promega). After 15 min each gel piece was re-swelled with 10 μL of ammonium bicarbonate 50 mM and incubated for 4 h at 37° C. After 4 h the reaction was stopped by adding 10 μL of trifluoroacetic acid 1% containing 1.5% (w/v) n-octyl-beta-D-glucopyranoside (OGP) (AppliChem). For the application of the samples, 4 μL of peptide solution were loaded onto an MTP Anchor Chip Target 600/384 (Bruker Daltonics) previously prepared with a saturated solution of matrix, alpha-cyano-4-hydroxy-cinnamic acid (alpha-HCCA) (Bruker Daltonics). An external calibration was performed by spotting on the 96 calibration positions of the Anchor Chip Target 1 μL of the peptide calibration standards (Bruker Daltonics) containing the following peptides: angiotensin 11 (1046.5420 Da), angiotensin I (1296.6853 Da), substance P (1347.7361 Da), bombesin (1619.8230 Da), ACTH clip 1-17 (2093.0868 Da), ACTH clip 18-39 (2465.1990 Da), somatostatin 28 (3147.4714 Da), and OGP 1.5% (w/v). Samples were analyzed in a MALDI-TOF-TOF spectrometer (Ultraflex, Bruker Daltonics) using an accelerating voltage of 25 kV for the Peptide Mass Fingerprint (PMF) mode. When necessary, MALDI-Post Source Decay (PSD) analysis was carried out using the LIFT special technique delivered by Bruker (the basic idea of LIFT is to lift the potential to fragment the selected peptides of interest). Peptide matching and protein searches were performed automatically with the MASCOT software. For the PMF search the parameters were the following: C-carbaimidomethyl (fixed modification), M-oxidation (variable modification), monoisotopic (mass value), 100 ppm (peptide mass tolerance), 1 (max missed cleavege), mammalia (taxonomy). Five matching peptides and at least 10% peptide coverage of the theoretical sequences was the minimal requirement for an identity assignment. For the MS/MS search (PSD) the parameters were the same except the peptide mass tolerance, which was 200 ppm. The identified Proteins™ individually, and only mouse proteins or highly homologous sequences from other mammalian species, like Homo sapiens or Rattus Norvegicus, having pl and Mw values close to the theoretical, were considered.

Immunohistochemistry:

Livers, dissected from EGF-overexpressing mice aged between 7-9 month, were fixed in 4% buffered paraformaldehyde and embedded in paraffin. 5 μm thick sections were deparaffinized and rehydrated through a descending alcohol series followed by a 4 min washing step in destilled H2O. Then, antigen retrieval was performed in citrate buffer (pH 6) by autoclaving the sections 15 min at 121° C. The Envision kit (Dako; Ham burg; Germany) was used for immunohistochemistry. The slides were rinsed with destilled H2O and after a 5 min incubation step in tris-buffered saline (washing buffer), endogenous peroxidase activity was blocked with DAKO Peroxidase blocking Reagent for 5 min followed by a second washing step. Thereafter, the sections were blocked for 10 min with protein-block serum free (Dako) and incubated with primary antibodies for 45 min. Details of antibody dilutions with washing buffer are given in table 1. In the case of goat primary antibodies a rabbit-anti-goat bridging antibody (Dako) was employed. Specifically, the bound primary antibodies or bridging antibodies were detected by use of labelled polymer HRP Anti-Rabbit secondary antibody (Envision Kit; Dako) and the immunoreactivity was visualized by DAKO Liquid DAB Substrate Chromogen System in an 5 min incubation. Finally, the sections were couterstained with Harris Haematoxylin for 2 min, dehydrated in an ascending alcohol series, coverslipped and examined under a light microscope. (Leica; Jülich; Germany)

| Antibody | Cat. Nr. | Dilution | Company |
|---|---|---|---|
| Antibody to GDI 2 | 10116-1-AP | 1:100 | Proteintech Europe |
| | | | Ltd.; Manchester, UK |
| Arginase II (H-64) | sc-20151 | 1:100 | Santa Cruz; |
| | | | Heidelberg; Germany |
| Antibody to CAPZA1 | 11806-1-AP | 1:400 | Proteintech Europe |
| | | | Ltd.; Manchester; UK |
| hnRNP L antibody | ab65049 | 1:1000 | Abcam; |
| | | | Cambridge; UK |
| Rabbit Anti-beta | ab52623 | 1:100 | Abcam; |
| Tubulin Monoclonal | | | Cambridge; UK |
| Antibody | | | |
| Amphiregulin (M-18) | sc-5797 | 1:150 | Santa Cruz; |
| | | | Heidelberg; Germany |
| Epiregulin (T-19) | sc-25232 | 1:150 | Abcam; |
| | | | Cambridge; UK |
| HNF-4α | sc-6556 | 1:250 | Santa Cruz; |
| | | | Heidelberg; Germany |

### Results

Image Analysis of Differentially Expressed Proteins:

Gels were scanned on a Molecular FX Scanner Bio-Rad at 100 μm resolution. Then, protein spots were imaged first automatically and then manually, and analyzed using the PDQuest™ software Bio-Rad (version 8.1). A total of 122 spots/proteins showed differences in expression levels between non-transgenic controls and HCC mice, and detailed information on these spots is listed in Table 1. Notable, FIG. 1 depicts some examples. Among them, 98 spots/proteins were statistically significantly regulated (p≦0.05); 62 of these spots/proteins were significantly upregulated (ratioHCC/control≧2), such as fibrinogen β, vimentin, Cu/Zn superoxide dismutase, and apolipoprotein E (FIG. 2-A, B, C, D), whereas 36 spots/proteins were significantly down-regulated (ratioHCC/control≦0.6), among them arginase 1, Dhdh protein, glutathione peroxidase 1, and predicted: agmatine ureohydrolase (FIG. 3-A, B, C, D) (see Table 1).

Identification of Proteins by MS Analysis:

Previously, a reference 2-DE map of mouse liver proteins has been created, consisting of more than 500 proteins [4]. Moreover, a reference 2-DE map of mouse serum proteins was reported in the same transgenic disease model and 25 serum proteins were detected to be differentially expressed, which are involved in a variety of cellular and metabolic pathways. Among them alpha-fetoprotein, clusterin, fibrinogen-α, fibrinogen-γ, serum amyloid component P, and some apolipoproteins were significally overexpressed [4]. In this work, a total of 122 differentially expressed spots/proteins were identified (Table 1), of which 98 were statistically significant. Interestingly, differentially expressed protein spots were found to be products of the same gene. They were: albumin (5 upregulated spots), alpha enolase (4 downregulated spots), apoliproptein A-I (2 upregulated spots), ATP synthase, H+ transporting, mitochondrial (2 downregulated spots), fibrinogen beta (2 upregulated spots), glycine N-methyltransferase (3 spots, in controls only), hsp60 (2 downregulated spot spots), nit protein 2 (2 downregulated spots), peroxiredoxin 6 (1 upregulated spot and 1 downregulated spot), and 4931406C07Rik (2 upregulated spots) (see Table 1). A total of 47 novel proteins were found to be HCC-associated. They are marked with an asterisk in Table 1. When disease regulated serum and liver proteins were compared, a total of 10 proteins were found to be regulated in common, therefore providing a direct link between regulated proteins of the tumor and serum proteome (Table 2). Among them, serum Afp was upregulated. Regulation of Afp and other proteins was confirmed by Western blot analysis. (FIG. 4-A, B, C, D, E).

An isoform of apolipoprotein 1 (gi|26345182), carboxylesterase precursor, fibrinogen alpha and fibrinogen gamma, were expressed exclusively in tumor samples. Fibrinogen beta was expressed exclusively in HCC serum, but displayed upregulation in HCC liver samples as well (ratio HCC/control=3.5). Apolipoprotein E was upregulated both in serum and liver samples, the ratio HCC/control being 2.2 and 3.9, respectively. Two spots of alpha-2-macroglobulin were upregulated in serum of HCC-bearing mice (spot 1: ratioHCC/control=1.8; spot 2: ratioHCC/control=3.2). In liver tissues, alpha-2-macroglobulin was exclusively expressed in tumor samples. Finally, serum amyloid component P was upregulated in serum up to 10-fold, and was expressed in HCC liver tissue only (Table 2).

Immunohistochemistry of Disease-Regulated Proteins:

To further confirm disease regulation in HCC a total of 8 proteins were selected for immunohistochemistry. Five of them were picked from the list of new but differently expressed proteins (see table 1). Additionally amphiregulin and epiregulin were chosen because of their importance as ligands in the EGF-signaling pathway. Also, HNF4α was selected for immunohistochemistry. This protein was shown to be significantly downregulated in HCC (74). As shown in FIGS. 5 and 6 immunohistochemistry confirms the regulation of the selected proteins. Indeed, arginase II, Capza1 GDI2 and amphiregulin were detected predominantly in the cytoplasmic compartment, whereas for hnRNPL and HNF4α nuclear expression was predominant. In tumors HNF4α was repressed and expression of tubulin β was observed particularly beneath the liver capsule.

### Discussion

This study aimed for an identification of novel disease regulated proteins in HCC. The proteome of healthy and liver tissues from HCC tumor-bearing mice was analyzed using the 2-DE technique coupled with MALDI-TOF MS/MS and 122 mouse liver proteins were identified to be differentially expressed. Here, 42 novel proteins are reported to be HCC disease-associated with prominent examples being discussed below.

Extra-Cellular Space or Secreted Proteins were Upregulated in HCC Livers

In this study, 62 upregulated proteins were identified. Among these, 18 (29%) were extra-cellular or secreted proteins, including albumin (main function transport), three apolipoproteins, apoE, apoA4, and apoA-I (cholesterol and lipid metabolic processes), α-, β-, and γ-fibrinogen, plasminogen, kininogen (main function in fibrinolysis and coagulation), interleukin 25 and interleukin 1 receptor antagonist protein (IL-1ra) (main functions immune and inflammatory responses and proliferation). Notably, an isoform of apoA1 has been proposed to be a serum marker of HCC [17]. Whereas the interleukin 1 receptor antagonist protein was immunohistochemically detected in tumor cells in approximately 70% of hepatocellular adenomas and carcinomas, eventhough early preneoplastic hepatocytic foci as well as normal hepatocytes surrounding the lesions were negative; consequently, this protein might be used to stage liver tumors. Additionally, RT-PCR analysis confirmed that mouse hepatic tumors contain both secreted and intracellular forms of IL-1ra [18]. Indeed, changes at the proteome level in serum have been used to monitor the effect of treatments applied to HCC patients [19].

Mitochondrial Proteins Involved in Biosynthetic Processes were Down-Regulated

Notably, in the results 14 (39%) mitochondrial proteins were down-regulated. This is in agreement with the results of Chignard and Wei Sun, which suggested that mitochondria were altered significantly during carcinogenesis, mitochondrial proteins being the second largest proportion of dysregulated proteins identified in HCC [20, 21]. These include NADH dehydrogenase (ubiquinone) 1 alpha subcomplex, 8, prohibitin (DNA replication), glutathione peroxidase 1 (induction of apoptosis by oxidative stress, response to oxidative stress), and argininosuccinate synthetase 1 (ASS) (urea cycle, amino acid biosynthetic process). This protein is the first of two enzymes to convert citrulline to arginine. This pathway allows cells to synthesize arginine from citrulline, making this amino acid non-essential for the growth of most mammalian cells. Previous studies demonstrated that several human tumor cell lines were auxotrophic for arginine due to an inability to express ASS [22-27]. In the study, as described herein, ASS was found in 15 out of 24 control gels, but none in tumor gels (see Table 1). Another protein that was found exclusively in control gels (23 out of 24) is the glycine N-methyltransferase (GNMT). This protein is strongly downregulated in HCC eventhough its expression is abundant in liver [28]. This multifunctional protein is involved in maintenance of genetic stability and frequently downregulated in HCC [29] (see Table 1).

### Newly Identified Disease-Related Proteins

We identified 122 mouse liver proteins to be differentially expressed. Some of these proteins have already been described in previous proteomic studies or are already known to be involved in hepatocarcinogenesis.

We therefore confirm earlier findings in an EGF-disease model of liver cancer. For these proteins references are reported in Table 1. The present study, however, compiles 42 newly identified and differentially expressed proteins which so far have not been described by previous proteomic studies to be involved in HCC. These proteins are marked with an asterisk in Table 1. Their functions cover a broad range spanning from metabolism to translation and signalling. For instance an elevated expression of proteins belonging to the carbohydrate metabolism was detected, like pyruvate kinase 3, aldolase 3 or alpha glucosidase 2 as well as of those involved in translation, like alanyl-tRNA synthetase (↑), eukaryotic translation elongation factor 2 (↑), or sars1 (↑). Proteins responsible for synthesis and degradation of lipids, steroids, fatty acids, and cholesterol, like the aldo-keto reductase family 1 (↑), butyryl coenzyme A synthetase 1 (↓), 2-hydroxyphytanoyl-CoA-lyase, enoyl coenzyme A hydratase 1, dihydrodiol dehydrogenase, lysophosphopholipase, mitochondrial acyl-CoA thioesterase 1 or farnesyl diphosphate synthetase (↑), which was also found to be upregulated in colonic cancers [71] are in contrast partially elevated in expression and partially downregulated. It was also found that RNA binding proteins like hnRNPL and Poly (rC) binding protein 2 were uniformly upregulated in HCC. Notably, hnRNPL was also upregulated in serum of patients diagnosed with HCC [72]. In contrast the ribosom-compononent rps12 and components of aminoacid metabolism like branched chain ketoacid dehydrogenase E1 or dimethyl glycine dehydrogease were repressed but proteasom components like psmd11 that was also found to be regulated in breast cancer [85] and p45/SUG was upregulated in tumor tissues. The results also show an enhanced expression of cytoskeletal proteins such as tubulin β 5 and Capza1 but immunohistochemical staining evidences differences in the localisation of these proteins beeing primarily associated with cells proximal to the capsule of the liver whereas Capza1 was strongly associated with the tumor foci. Also GDI2, a protein that functions in the cycling of Rab GTPases and arginase II, an non liver isoform of the urea cycle protein, were upregulated in HCC bearing mice (see FIG. 5). Notably the regulation of arginase II in lung cancer was already reported [73]. Moreover the actin-binding protein LASP1 was only found in tumorous tissues.

It is known to be upregulated in breast cancer [86] and could be responsible for migration of cancer cells. [87]. Other proteins with elevated expression are Kininogen, a part of the blood coagulation system and the precurser of kinin, and Pdia4 a disulfide bond isomerase, whereas Serpinb1a a serinproteinsase inhibitor was found to be downregulated in HCC mice. Also the calcium binding protein Sorcin and Nucleobindin 1, a protein that may play a role in calcium mediated signaling, could be a proteins of further interest. Sorcin was found to be correlated with multidrug resistance in human leukemia cells [88] were as nucleobindin1 is a potential biomarker for coloncancer [89]. Transthyretin, also upregulated in HCC, is a transport protein of thyroid hormons and synthesized by the liver. Interestingly its regulation was shown in thyroid cancer [90]. Noteworthy, among the newly identified 42 HCC-related proteins are the following: v-crk sarcoma virus CT10, an oncoprotein involved in the intracellular signaling cascade and in the activation of the phosphoinositide 3-kinase (PI3K)/AKT pathway [30], and the 170 kDa glucose-regulated protein GRP170, an endoplasmic reticulum lumenal glycoprotein that may play a role in immunoglobulin folding. In fact, GRP170 was found to be precipitated with immunoglobulin in four different B cell hybridomas [31]. A summary of the biological functions and their previous reported tumor association is given in table 3. In a previous study to map the serum proteome of hepatocellular carcinoma induced by targeted overexpression of Egf to liver cells of transgenic mice, many immunoglobulins were found to be repressed or absent in serum samples of tumor-bearin mice, like the Ig K and L classes [4]. Whether GRP170 is responsible for the repression of immunoglobulins in the sera of Egf tumor-bearing mice needs further studies. (See table 3)

To identify disease proteins regulated in EGF-HCC-bearing transgenic mice, a proteomic approach has been used, as described herein, that consisted of two-dimensional electrophoresis (2-DE) and MALDI-MS/MS. A total of 98 proteins showed significant differences in expression levels between non-transgenic controls and HCC-bearing mice; 62 proteins were upregulated, whereas 36 proteins were downregulated. Although some of these proteins have already been described in previous proteomic studies or are already known to be involved in hepatocarcinogenesis, this work reports 42 new proteins differentially expressed in HCC-bearing mice. Additionally, regulation of the proteins was confirmed by immunohistochemistry and western blotting. Moreover, the results were compared and correlate with those of the previous work of the inventors, where sera of EGF induced HCC-bearing mice and of non-transgenic controls were studied. Thus, a total of ten disease-regulated liver proteins were identified as commonly regulated in sera and tumor tissue of HCC-bearing mice.

In conclusion, the study within the context of the invention identified a total of 42 proteins which so far have been unknown to be regulated in hepatocellular carcinoma and may enable an improved understanding of HCC disease. Many previously reported HCC-related proteins were also identified in the study. Serum and liver proteomes of tumor-bearing mice were studied and compared. Ten proteins were found to have the same regulation both in sera and liver tissue of the same EGF-HCC mouse model, providing a direct link between regulated proteins of the tumor and serum proteomes. Obviously, these are highly interesting biomarkers for HCC.

### FIGURE CAPTIONS

FIG. 1. Upregulation and downregulation of some of the 122 deregulated mouse liver proteins. Spot 1: glycine N-methyltransferase, identified in control samples only; Spot 2: peroxiredoxin 6, upregulated in tumor samples (ratioHCC/control=2.3); Spot 3: peroxiredoxin 6, downregulated in tumor samples (ratioHCC/control=0.3); Spot 4: lysophospholipase 1, downregulated in tumor samples (ratioHCC/control=0.2); Spot 5: hypothetical protein LOC68347, downregulated in tumor samples (ratioHCC/control=0.4); Spot 6: glutathione peroxidase 1, downregulated in tumor samples (ratioHCC/control=0.3).

FIG. 2. Some of the 62 upregulated mouse liver proteins: fibrinogen 8 (FIG. 2-A), vimentin (FIG. 2-B), Cu/Zn superoxide dismutase (FIG. 2-C), and apolipoprotein E (FIG. 2-D).

FIG. 3. Some of the 36 downregulated mouse liver proteins: arginase 1 (FIG. 3-A), Dhdh protein (FIG. 3-B), glutathione peroxidase 1 (FIG. 3-C), and predicted: agmatine ureohydrolase (FIG. 3-D).

FIG. 4. Western blots of alpha-fetoprotein (A), fibrinogen gamma (B), serum amyloid component P (C), epidermal growth factor (D), and apolipoprootein M, which was identified in serum samples only (E); C=control, T=tumor.

FIG. 5 Immunohistochemical stainings of Arginase II, Capza1, GDI 2 and Tubulin β Conrols were treated with washing buffer instead of primary antibody.

FIG. 6a-b Immunohistochemical stainings of hnRNPL, Amphiregulin HNF4a and Epiregulin Conrols were treated with washing buffer instead of primary antibody. For amphiregulin the control staining was done with blocking peptide.

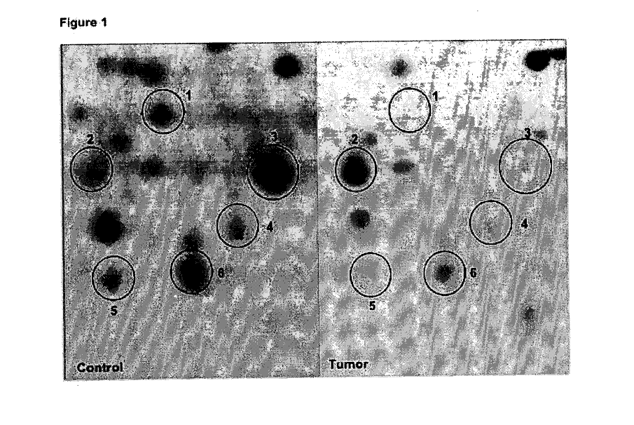

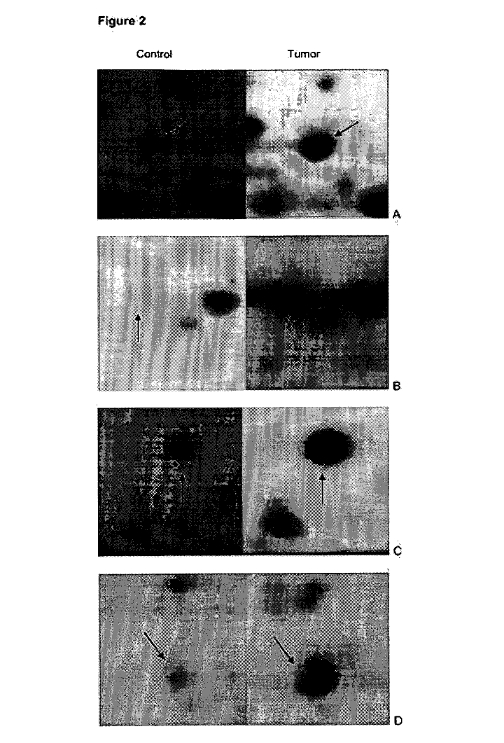

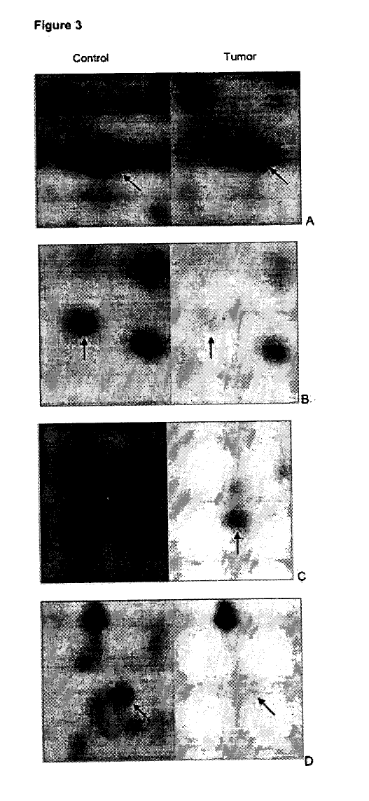

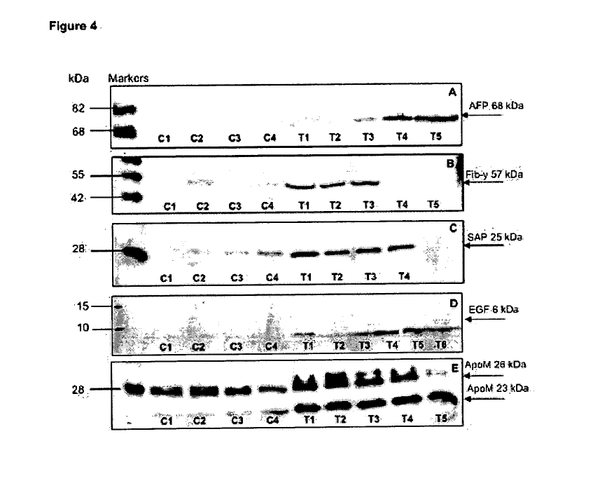

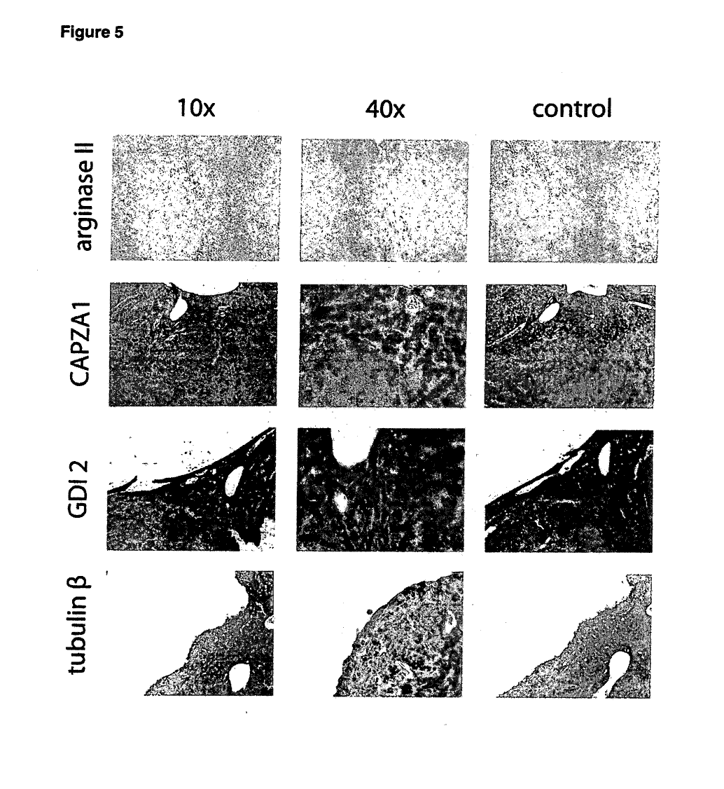

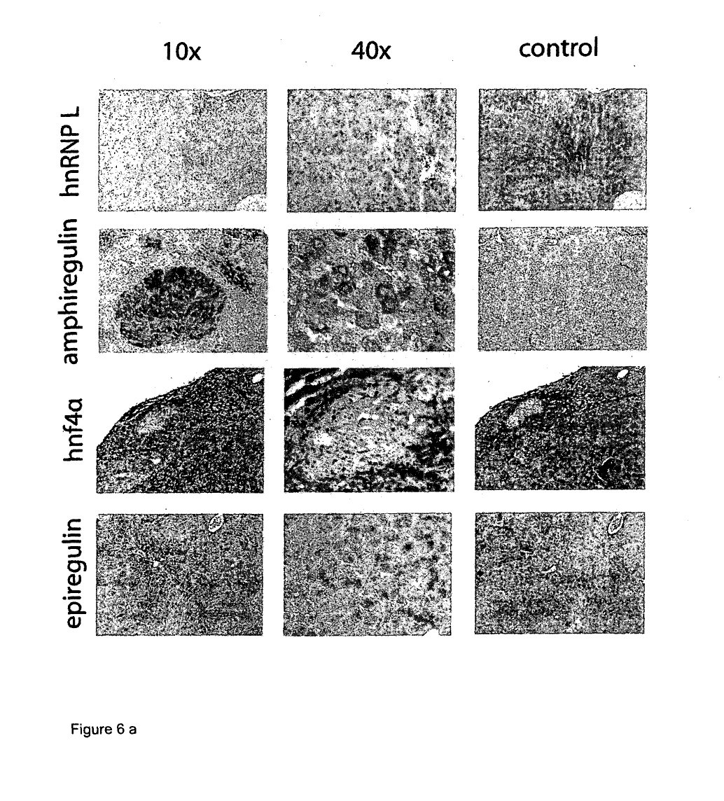

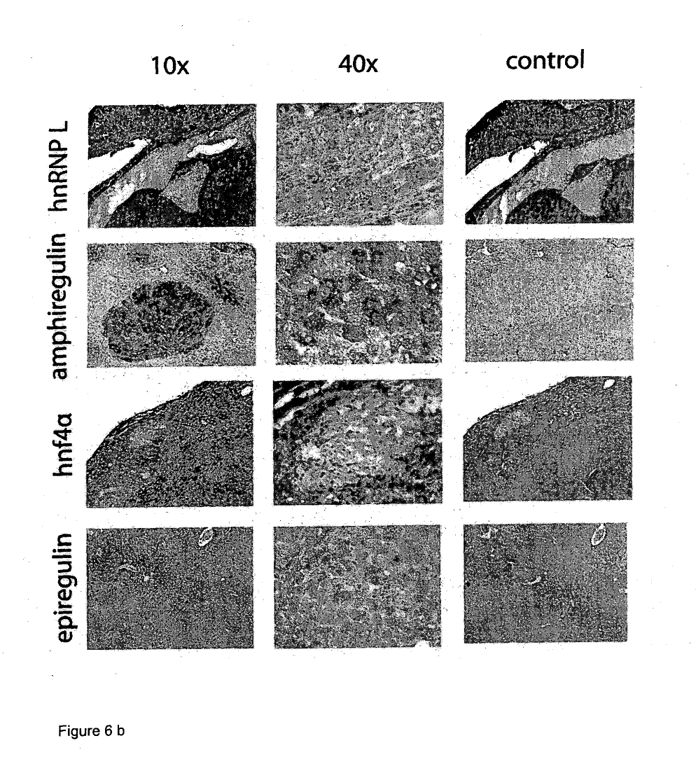

Table 1. List of the 122 differentially regulated proteins. The proteins are sorted in alphabetical order, and the NCB, annotation is given in the accession number column. Molecular weight, pl, and MASCOT scores are also reported in the table. The column “Gels” indicates in how many different gels of the total 48 cut gels each protein was identified; the columns “C” (C=control) and “T” (T=tumor) indicate in how many control and tumor gels/samples each protein was identified; the columns “LB2” and “LB3” (LB=lysis buffer) indicate how many times each protein was identified in LB2 and/or LB3. Data like p-value, ratio T/C, and cellular location are also reported in the table. The column “references” reports citations for those proteins which have already been described to be HCC-associated proteins. * Proteins which so far have been unknown to be regulated in hepatocellular carcinoma Abbreviations: C, cytosol; Ck, cytoskeleton; M, mitochondria; N, nucleus; P, peroxisome; ES, extracellular space; ER, endoplasmic reticulum; G, golgi; L, lysosome; MEM, membrane; MM, mitochondrial matrix; S, secreted.

Table 2 The ten differentially expressed mouse proteins common to serum and liver samples. For each sample the ratioHCC/control is given.

Table 3 Biological function and tumor association of new and differentially regulated proteins.

Table 4: Overview of the 122 proteins in comparison with the state of the art, wherein “x” denotes “References” ([ . . . ]) with regard to “HCC” or “Other cancer” or the protein is “unknown”, so far, in the context of cancer.

### REFERENCES

(1) Gazzana, G.; Borlak, J. J Proteome. Res 2007, 6(8), 3143-3151.
(2) Cha, C.; DeMatteo, R. P.; Blumgart, L. H. J Clin Gastroenterol 2002, 35(5 Suppl 2), S130-S137.
(3) Yano, S.; Kondo, K.; Yamaguchi, M.; Richmond, G.; Hutchison, M.; Wakeling, A.; Averbuch, S.; Wadsworth, P. Anticancer Res 2003, 23(5A), 3639-3650.
(4) Gazzana, G.; Borlak, J. J Proteome. Res 2008, 7(3):928-937.
(5) Melle, C.; Kaufmann, R.; Hommann, M.; Bleul, A.; Driesch, D.; Ernst, G.; Von Eggeling, F. Int J Oncol 2004, 24(4), 885-891.
(6) Melle, C.; Ernst, G.; Scheibner, O.; Kaufmann, R.; Schimmel, B.; Bleul, A.; Settmacher, U.; Hommann, M.; Claussen, U.; Von Eggeling, F. J Proteome. Res 2007, 6(1), 306-315.
(7) Izzo, F.; Cremona, F.; Delrio, P.; Leonardi, E.; Castello, G.; Pignata, S.; Daniele, B.; Curley, S. A. Ann Surg. Oncol 1999, 6(2), 178-185.
(8) Fujii, K.; Kondo, T.; Yokoo, H.; Yamada, T.; Iwatsuki, K.; Hirohashi, S. Proteomics. 2005, 5(5), 1411-1422.
(9) Seow, T. K.; Ong, S. E.; Liang, R. C.; Ren, E. C.; Chan, L.; Ou, K.; Chung, M. C. Electrophoresis 2000, 21(9), 1787-1813.
(10) Ou, K.; Seow, T. K.; Liang, R. C.; Ong, S. E.; Chung, M. C. Electrophoresis 2001, 22(13), 2804-2811.
(11) Liang, R. C.; Neo, J. C.; Lo, S. L.; Tan, G. S.; Seow, T. K.; Chung, M. C. J. Chromatogr. B Analyt. Technol. Biomed Life Sci 2002, 771(1-2), 303-328.
(12) Cui, J. F.; Liu, Y. K.; Pan, B. S.; Song, H. Y.; Zhang, Y.; Sun, R. X.; Chen, J.; Feng, J. T.; Tang, Z. Y.; Yu, Y. L.; Shen, H. L.; Yang, P. Y. J Cancer Res Clin Oncol 2004, 130(10), 615-622.
(13) Yang, Z. F.; Ho, D. W.; Lam, C. T.; Luk, J. M.; Lum, C. T.; Yu, W. C.; Poon, R. T.; Fan, S. T. Cancer Res 2005, 65(1), 219-225.
(14) Block, T. M.; Comunale, M. A.; Lowman, M.; Steel, L. F.; Romano, P. R.; Fimmel, C.; Tennant, B. C.; London, W. T.; Evans, A. A.; Blumberg, B. S.; Dwek, R. A.; Mattu, T. S.; Mehta, A. S. Proc Natl Acad Sci U.S.A 2005, 102(3), 779-784.
(15) Cui, F.; Wang, Y.; Wang, J.; Wei, K.; Hu, J.; Liu, F.; Wang, H.; Zhao, X.; Zhang, X.; Yang, X. Proteomics. 2006, 6(2), 498-504.
(16) Ding, S. J.; Li, Y.; Tan, Y. X.; Jiang, M. R.; Tian, B.; Liu, Y. K.; Shao, X. X.; Ye, S. L.; Wu, J. R.; Zeng, R.; Wang, H. Y.; Tang, Z. Y.; Xia, Q. C. Mol Cell Proteomics. 2004, 3(1), 73-81.
(17) Steel, L. F.; Shumpert, D.; Trotter, M.; Seeholzer, S. H.; Evans, A. A.; London, W. T.; Dwek, R.; Block, T. M. Proteomics. 2003, 3(5), 601-609.
(18) Yamada, Y.; Karasaki, H.; Matsushima, K.; Lee, G. H.; Ogawa, K. Lab Invest 1999, 79(9), 1059-1067.
(19) Kawakami, T.; Hoshida, Y.; Kanai, F.; Tanaka, Y.; Tateishi, K.; Ikenoue, T.; Obi, S.; Sato, S.; Teratani, T.; Shiina, S.; Kawabe, T.; Suzuki, T.; Hatano, N.; Taniguchi, H.; Omata, M. Proteomics. 2005, 5(16), 4287-4295.
(20) Chignard, N.; Beretta, L. Gastroenterology 2004, 127(5 Suppl 1), S120-S125.
(21) Sun, W.; Xing, B.; Sun, Y.; Du, X.; Lu, M.; Hao, C.; Lu, Z.; Mi, W.; Wu, S.; Wei, H.; Gao, X.; Zhu, Y.; Jiang, Y.; Qian, X.; He, F. Mol Cell Proteomics. 2007, 6(10), 1798-1808.
(22) Dillon, B. J.; Prieto, V. G.; Curley, S. A.; Ensor, C. M.; Holtsberg, F. W.; Bomalaski, J. S.; Clark, M. A. Cancer 2004, 100(4), 826-833.
(23) Cheng, P. N.; Lam, T. L.; Lam, W. M.; Tsui, S. M.; Cheng, A. W.; Lo, W. H.; Leung, Y. C. Cancer Res 2007, 67(1), 309-317.
(24) Yoon, C. Y.; Shim, Y. J.; Kim, E. H.; Lee, J. H.; Won, N. H.; Kim, J. H.; Park, I. S.; Yoon, D. K.; Min, B. H. Int J Cancer 2007, 120(4), 897-905.
(25) Svetlov, S. I.; Xiang, Y.; Oli, M. W.; Foley, D. P.; Huang, G.; Hayes, R. L.; Ottens, A. K.; Wang, K. K. Biomarkers 2006, 11(4), 355-369.
(26) Feun, L.; Savaraj, N. Expert Opin. Investig. Drugs 2006, 15(7), 815-822.
(27) Wheatley, D. N.; Campbell, E. Br J Cancer 2003, 89(3), 573-576.
(28) Velichkova, P.; Himo, F. J Phys. Chem B 2005, 109(16), 8216-8219.
(29) Tseng, T. L.; Shih, Y. P.; Huang, Y. C.; Wang, C. K.; Chen, P. H.; Chang, J. G.; Yeh, K. T.; Chen, Y. M.; Buetow, K. H. Cancer Res 2003, 63(3), 647-654.
(30) Akagi, T.; Murata, K.; Shishido, T.; Hanafusa, H. Mol Cell Biol 2002, 22(20), 7015-7023.
(31) Lin, H. Y.; Masso-Welch, P.; Di, Y. P.; Cai, J. W.; Shen, J. W.; Subjeck, J. R. Mol Biol Cell 1993, 4(11), 1109-1119.
(32) Easton, D. P.; Kaneko, Y.; Subjeck, J. R. Cell Stress. Chaperones. 2000, 5(4), 276-290.
(33) Liang, R. C.; Neo, J. C.; Lo, S. L.; Tan, G. S.; Seow, T. K.; Chung, M. C. J Chromatogr. B Analyt. Technol. Biomed Life Sci 2002, 771(1-2), 303-328.
(34) Blanc, J. F.; Lalanne, C.; Plomion, C.; Schmitter, J. M.; Bathany, K.; Gion, J. M.; Bioulac-Sage, P.; Balabaud, C.; Bonneu, M.; Rosenbaum, J. Proteomics. 2005, 5(14), 3778-3789.
(35) Yang, M. H.; Tyan, Y. C.; Jong, S. B.; Huang, Y. F.; Liao, P. C.; Wang, M. C. Anal. Bioanal. Chem 2007, 388(3), 637-643.
(36) Shen, H.; Cheng, G.; Fan, H.; Zhang, J.; Zhang, X.; Lu, H.; Liu, C.; Sun, F.; Jin, H.; Xu, X.; Xu, G.; Wang, S.; Fang, C.; Bao, H.; Wang, Y.; Wang, J.; Zhong, H.; Yu, Z.; Liu, Y.; Tang, Z.; Yang, P. Proteomics. 2006, 6(2), 528-537.
(37) Melle, C.; Ernst, G.; Scheibner, O.; Kaufmann, R.; Schimmel, B.; Bleul, A.; Settmacher, U.; Hommann, M.; Claussen, U.; Von Eggeling, F. J Proteome. Res 2007, 6(1), 306-315.
(38) Takashima, M.; Kuramitsu, Y.; Yokoyama, Y.; lizuka, N.; Fujimoto, M.; Nishisaka, T.; Okita, K.; Oka, M.; Nakamura, K. Proteomics. 2005, 5(6), 1686-1692.
(39) Wu, N.; Zhang, W.; Yang, Y.; Liang, Y. L.; Wang, L. Y.; Jin, J. W.; Cai, X. M.; Zha, X. L. Proteomics. 2006, 6(22), 6095-6106.
(40) He, Q. Y.; Lau, G. K.; Zhou, Y.; Yuen, S. T.; Lin, M. C.; Kung, H. F.; Chiu, J. F. Proteomics. 2003, 3(5), 666-674.
(41) Yokoyama, Y.; Kuramitsu, Y.; Takashima, M.; lizuka, N.; Terai, S.; Oka, M.; Nakamura, K.; Okita, K.; Sakaida, I. Int J Oncol 2006, 28(3), 625-631.
(42) Ai, J.; Tan, Y.; Ying, W.; Hong, Y.; Liu, S.; Wu, M.; Qian, X.; Wang, H. Proteomics. 2006, 6(2), 538-546.
(43) Liang, C. R.; Leow, C. K.; Neo, J. C.; Tan, G. S.; Lo, S. L.; Lim, J. W.; Seow, T. K.; Lai, P. B.; Chung, M. C. Proteomics. 2005, 5(8), 2258-2271.
(44) Kuramitsu, Y.; Nakamura, K. Expert Rev Proteomics. 2005, 2(4), 589-601.
(45) Yokoyama, Y.; Kuramitsu, Y.; Takashima, M.; lizuka, N.; Toda, T.; Terai, S.; Sakaida, I.; Oka, M.; Nakamura, K.; Okita, K. Proteomics. 2004, 4(7), 2111-2116.
(46) Li, C.; Hong, Y.; Tan, Y. X.; Zhou, H.; Ai, J. H.; Li, S. J.; Zhang, L.; Xia, Q. C.; Wu, J. R.; Wang, H. Y.; Zeng, R. Mol Cell Proteomics. 2004, 3(4), 399-409. (47) Capuano, F.; Varone, D.; D'Eri, N.; Russo, E.; Tommasi, S.; Montemurro, S.; Prete, F.; Papa, S. Biochem Mol Biol Int 1996, 38(5), 1013-1022.
(48) Xu, X. R.; Huang, J.; Xu, Z. G.; Qian, B. Z.; Zhu, Z. D.; Yan, Q.; Cai, T.; Zhang, X.; Xiao, H. S.; Qu, J.; Liu, F.; Huang, Q. H.; Cheng, Z. H.; Li, N. G.; Du, J. J.; Hu, W.; Shen, K. T.; Lu, G.; Fu, G.; Zhong, M.; Xu, S. H.; Gu, W. Y.; Huang, W.; Zhao, X. T.; Hu, G. X.; Gu, J. R.; Chen, Z.; Han, Z. G. Proc Natl Acad Sci U.S.A 2001, 98(26), 15089-15094.
(49) Kinoshita, M.; Miyata, M. Hepatology 2002, 36(2), 433-438.
(50) Lim, S. O.; Park, S. J.; Kim, W.; Park, S. G.; Kim, H. J.; Kim, Y. I.; Sohn, T. S.; Noh, J. H.; Jung, G. Biochem Biophys. Res Commun. 2002, 291(4), 1031-1037.
(51) Tada, M.; Yokosuka, O.; Fukai, K.; Chiba, T.; Imazeki, F.; Tokuhisa, T.; Saisho, H. J Hepatol 2005, 42(4), 511-519.
(52) Amacher, D. E.; Adler, R.; Herath, A.; Townsend, R. R. Clin Chem 2005, 51(10), 1796-1803.
(53) Novoselov, S. V.; Calvisi, D. F.; Labunskyy, V. M.; Factor, V. M.; Carlson, B. A.; Fomenko, D. E.; Moustafa, M. E.; Hatfield, D. L.; Gladyshev, V. N. Oncogene 2005, 24(54), 8003-8011.
(54) Gladyshev, V. N.; Factor, V. M.; Housseau, F.; Hatfield, D. L. Biochem Biophys. Res Commun. 1998, 251(2), 488-493.
(55) Baker, R. D.; Baker, S. S.; LaRosa, K.; Whitney, C.; Newburger, P. E. Arch Biochem Biophys. 1993, 304(1), 53-57.
(56) Liu, H. H.; Chen, K. H.; Shih, Y. P.; Lui, W. Y.; Wong, F. H.; Chen, Y. M. J Biomed Sci 2003, 10(1), 87-97.
(57) Lee, C. L.; Hsiao, H. H.; Lin, C. W.; Wu, S. P.; Huang, S. Y.; Wu, C. Y.; Wang, A. H.; Khoo, K. H. Proteomics. 2003, 3(12), 2472-2486.
(58) Zhao, Q.; Zhao, M.; Zhang, C. Zhongguo Yi. Xue. Ke. Xue. Yuan Xue. Bao. 1994, 16(2), 135-139.
(59) Liu, Y.; Zhu, X.; Zhu, J.; Liao, S.; Tang, Q.; Liu, K.; Guan, X.; Zhang, J.; Feng, Z. Oncol Rep 2007, 18(4), 943-951.
(60) Raynes, J. G.; Bevan, S. Agents Actions 1993, 38 Spec No C66-C68.
(61) Bevan, S.; Raynes, J. G. J Immunol 1991, 147(8), 2574-2578.
(62) Stienstra, R.; Mandard, S.; Tan, N. S.; Wahli, W.; Trautwein, C.; Richardson, T. A.; Lichtenauer-Kaligis, E.; Kersten, S.; Muller, M. J Hepatol 2007, 46(5), 869-877.
(63) Dragani, T. A.; Manenti, G.; Sacchi, M. R.; Colombo, B. M.; Della, P. G. Mol Carcinog. 1989, 2(6), 355-360.
(64) Seow, T. K.; Liang, R. C.; Leow, C. K.; Chung, M. C. Proteomics. 2001, 1(10), 1249-1263.
(65) Schmitz, V.; Raskopf, E.; Gonzalez-Carmona, M. A.; Vogt, A.; Rabe, C.; Leifeld, L.; Kornek, M.; Sauerbruch, T.; Caselmann, W. H. Gut 2007, 56(2), 271-278.
(66) Tao, K. S.; Dou, K. F.; Wu, X. A. World J Gastroenterol 2004, 10 (10), 1421-1424.
(67) Kim, S.; Park, H. S.; Son, H. J.; Moon, W. S. Korean J Hepatol 2004, 10(1), 62-72.
(68) Fielden, M. R.; Brennan, R.; Gollub, J. Toxicol Sci 2007, 99(1), 90-100.
(69) Chan, K. Y.; Lai, P. B.; Squire, J. A.; Beheshti, B.; Wong, N. L.; Sy, S. M.; Wong, N. Mod. Pathol 2006, 19(12), 1546-1554.
(70) Egawa, T.; Ito, H.; Nakamura, H.; Yamamoto, H.; Kishimoto, S. Biochem Int 1992, 28(3), 551-557.

(71). Notarnicola, M.; Cavallini, A.; Bifulco, M.; Tecce, M F.; Eletto, D, Di Leo A, Montemurro, S.; Laezza, C.; Caruso, M. G. Oncology 2004, 67(5-6), 351-358.
(72) Looi, K. S.; Nakayasu, E. S.; Diaz, R. A.; Tan, E. M.; Almeida, I. C., Zhang, J. Journal of proteome research 2008, 7(9), 4004-4012.
(73) Rotondo, R.; Mastracci, L.; Piazza, T.; Barisione, G,; Fabbi, M.; Cassanello, M; Costa, R.; Morandi, B.; Astigiano, S.; Cesario, A.; Sormani, M. P.; Ferlazzo, G,; Grossi, F.; Ratto, G. B.; Ferrini, S.; Frumento, G. Int. J. Cancer 2008, 123 (5), 1108-1116.
(74) Lazarevich, N. L.; Cheremnova, O. A.; Varga, E. V.; Ovchinnikov, D. A.; Kudrjavtseva, EA.; Morozova, O. V.; Fleishman, D. I.; Engelhardt, N. V.; Duncan, S. A. Hepatology. 2004, 39(4), 1038-47.
(75) Lin Y. H.; Park Z. Y.; Lin D.; Brahmbhatt A. A.; Rio M. C.; Yates J. R. 3rd; Klemke R. L. J. Cell Biol. 2004; 165(3):421-32.
(76) Foulon, V.; Antonenkov, V. D.; Croes, K.; Waelkens, E.; Mannaerts, G. P.; Van eldhoven, P. P.; Casteels, M. Proceedings of the national Academy of science U.S.A. 1999, 96(18), 10039-44.
(77) Wu, G.; Morris, S. M., Jr. Biochemical Journal 1998, 336 (Pt 1), 1-17.
(78) Lin, P.; Le-Niculescu, H.; Hofineister, R.; McCaffery, J. M.; Jin, M.; Hennemann, H.; McQuistan, T.; Vries, L. and Gist Farquhar M. Cell Biol. 1998, 141(7), 1515-1527.
(79) Meyers, M. B.; Fischer, A.; Sun, Y.; Lopes, C. M. B.; Rohacs, T.; Nakamura, T. Y.; Zhou, Y.; Lee, P. C.; Altschuld, R. A.; McCune, S. A.; Coetzee, W. A. and Fishman G. I. J Biol Chem 2003, 278 (31), 28865-71.
(80) Looi, K. S.; Nakayasu, E. S.; Diaz, R. A.; Tan, E. M.; Almeida, I. C.; Zhang, J. Y. Journal of proteome research 2008, 7 (9), 4004-4012.
(81) Teramotoa, R.; Minagawab, H.; Honda, M.; Miyazakia, K.; Tabusea, Y.; Kamijob, K.; Uedac, T. and Kanekoc, S. Biochimica et Biophysica Acta (BBA)—Proteins & Proteomics, 2008, 1784 (5),764-772.
(82) Miller, L. M.; Menthena, A.; Chatterjee, C.; Verdier-Pinard, P.; Novikoff, P. M.; Horwitz, S. B.; Angeletti, R. H. Biochemistry 2008, 47 (28), 7572-7582.
(83) Dallmann, K.; Junker, H.; Balabanov, S.; Zimmermann, U.; Giebel, J.; Walther, R. Int. J. Cancer 2004, 108 (3), 342-347.
(84) Glassman, R. H.; Hempstead, B. L.; Staiano-Coico, L.; Steiner, M. G.; Hanafusa, H.; Birge, R B. Cell Death and Differentiation 1997, 4 (1), 82-93.
(85) Deng, S.; Zhou, H.; Xiong, R.; Lu, Y.; Yan, D.; Xing, T.; Dong, L.; Tang, E.; Yang, H. Breast Cancer Res Treat. 2007, 104(1), 21-30
(86) Grunewald, T. G.; Kammerer, U.; Kapp, M.; Eck, M.; Dietl, J.; Butt, E.; Honig, A.; BMC Cancer. 2007; 7, 198.
(87) Lin, Y. H.; Park, Z. Y.; Lin, D.; Brahmbhatt, A. A.; Rio, M. C.; Yates, J. R. 3rd; Klemke, R. L. J Cell Biol. 2004, 165(3), 421-32.
(88) Zhou, Y.; Xu, Y.; Tan, Y.; Qi, J.; Xiao, Y.; Yang, C.; Zhu, Z.; Xiong, D. Leuk Res. 2006, 30(4), 469-76.
(89) Chen, Y.; Lin, P.; Qiu, S.; Peng, X. X.; Looi, K.; Farquhar, M. G.; Zhang, J. Y. Int J Oncol. 2007, 30(5), 1137-44.
(90) Giusti, L.; Iacconi, P.; Ciregia, F.; Giannaccini, G.; Donatini, G. L.; Basolo, F.; Miccoli, P.; Pinchera, A.; Lucacchini, A. J Proteome Res. 2008, 7(9), 4079-88.
(91) Li, L.; Chen S. H.; Yu, C. H.; Li, Y. M.; Wang, S. Q. J Proteome Res. 2008, 7(2), 611-20.
(92) Khalil, A A. Cancer Sci. 2007, 98(2), 201-13.
(93) Kim, H. K.; Park, W S.; Kang, S. H.; Warda, M.; Kim, N.; Ko, J. H.; Prince, Ael-B.; Han, J. Am J Physiol Cell Physiol. 2007, 293(2), C761-71.
(94) Cucchiarelli, V.; Hiser, L.; Smith, H.; Frankfurter, A.; Spano, A.; Correia, J. J.; Lobert, S. Cell Motil Cytoskeleton. 2008, 65(8), 675-85.
(95) Deng, S. S.; Xing, T. Y.; Zhou, H. Y.; Xiong, R. H.; Lu, Y. G.; Wen, B.; Liu, S. Q, Yang, H. J. Genomics Proteomics Bioinformatics. 2006 August; 4(3), 165-72.
(96) Chang, H. C.; Chen, Y. L.; Chan, C. P.; Yeh, K. T.; Kuo S. J.; Ko, C. J.; Fang, H. Y.; Dig Dis Sci. 2008. [Epub ahead of print]
(97) Al-Dhaheri, M. H.; Shah, Y. M.; Basrur, V.; Pind, S.; Rowan, B. G. Steroids. 2006, 71(11-12),966-78.
(98) Dingemans, A. M.; van Ark-Otte, J.; van der Valk, P.; Apolinario, R. M.; Scheper, R. J.; Postmus, P. E.; Giaccone, G. Ann Oncol. 1996, 7(6), 625-30.
(99) Aoki, S.; Ishikura, S.; Asada, Y.; Usami, N.; Hara, A. Chem Biol Interact. 2001, 130-132(1-3), 775-84.
(100) Meyers, M. B.; Fischer, A.; Sun, Y. J.; Lopes, C. M.; Rohacs, T.; Nakamura, T. Y.; Zhou, Y. Y.; Lee, P. C.; Altschuld, R. A.; McCune, S. A.; Coetzee, W. A.; Fishman, G. I. J Biol. Chem 2003, 278(31), 28865-28871, August 1.

The characteristics of the invention being disclosed in the preceding description, the subsequent drawings and claims can be of importance both singularly and in arbitrary combination for the implementation of the invention in its different embodiments.

The foregoing description of preferred embodiments of the invention has been presented for the purposes of illustration and description. It is not intended to be exhaustive or to limit the invention to the precise form described, and many modifications and variations are possible in light of the teaching above. The embodiments were chosen and described in order to best explain the principles of the invention and its practical applications to thereby enable others skilled in the art to best utilize the invention in various embodiments and with various modifications as are suited to the particular use contemplated invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

TABLE 1

| | | Accession | | | Mascot | | | | | | | Ratio | Cellular | |
|---|---|---|---|---|---|---|---|---|---|---|---|---|---|---|
| No. | Protein | number | Mr | pl | score | Gels | C | T | LB2 | LB3 | p-value | T/C | location | References |
| 1* | 170 kDa glucose regulated | gi\|7643979 | 111 | 5 | 214 | 10 | 0 | 10 | 8 | 2 | | T | ER, ES | [31, 32] |
| | protein GRP170 precursor | | | | | | | | | | | | | |
| 2* | 2-hydroxyphytanoyl-CoA | gi\|18204150 | 63.6 | 5.9 | 261 | 32 | 19 | 13 | 22 | 10 | 0.027 | 0.38 | P | |
| | lyase | | | | | | | | | | | | | |
| 3 | 3-phosphoglycerate | gi\|52353955 | 56.6 | 6 | 204 | 14 | 0 | 14 | 11 | 3 | | T | | [33] |
| | dehydrogenase | | | | | | | | | | | | | |
| 4* | 4931406C07Rik (Ester | gi\|71059921 | 35 | 5.8 | 184 | 35 | 17 | 18 | 24 | 11 | 0.033 | 1.5 | N | |
| | hydrolase C11orf54 | | | | | | | | | | | | | |
| | homolog) (spot 4342) | | | | | | | | | | | | | |
| 5* | 4931406C07Rik (Ester | gi\|71059921 | 35 | 5.8 | 195 | 33 | 15 | 18 | 22 | 11 | 0.244 | 1.9 | N | |
| | hydrolase C11orf54 | | | | | | | | | | | | | |
| | homolog) (spot 4349) | | | | | | | | | | | | | |
| 6 | Acylpeptide hydrolase; N- | gi\|22122789 | 79.9 | 5.2 | 182 | 5 | 0 | 5 | 0 | 5 | | T | | [34] |
| | acylaminoacyl peptide | | | | | | | | | | | | | |
| | hydrolase | | | | | | | | | | | | | |
| 7* | Akr1c12 protein | gi\|15215042 | 37 | 6.1 | 135 | 24 | 14 | 10 | 15 | 9 | 0.301 | 1.9 | | |
| 8 | Akr1c18 protein (aldo-keto | gi\|19527284 | 37.2 | 5.9 | 189 | 11 | 0 | 11 | 5 | 6 | | T | C | [21] |
| | reductase family 1, | | | | | | | | | | | | | |
| | member C18) | | | | | | | | | | | | | |
| 9* | Alanyl-tRNA synthetase | gi\|34610207 | 106.9 | 5.4 | 178 | 4 | 0 | 4 | 0 | 4 | | T | C | |
| 10 | Albumin 1 (spot 3707) | gi\|33859506 | 68.7 | 5.7 | 444 | 43 | 21 | 22 | 31 | 12 | 0.068 | 3.88 | C, ES | [21, 35] |
| 11 | Albumin 1 (spot 3712) | gi\|33859506 | 68.7 | 5.7 | 416 | 40 | 20 | 20 | 30 | 10 | 0.25 | 2.04 | C, ES | [21, 35] |
| 12 | Albumin 1 (spot 4506) | gi\|33859506 | 68.7 | 5.7 | 423 | 40 | 20 | 20 | 31 | 9 | | T | C, ES | [21, 35] |
| 13 | Albumin 1 (spot 4702) | gi\|33859506 | 68.7 | 5.7 | 355 | 42 | 20 | 22 | 31 | 11 | 0.069 | 1.85 | C, ES | [21, 35] |
| 14 | Albumin 1 (spot 5509) | gi\|33859506 | 68.7 | 5.7 | 473 | 38 | 18 | 20 | 30 | 8 | 0.392 | 3.59 | C, ES | [21, 35] |
| 15* | Aldo-keto reductase family | gi\|19527294 | 37.2 | 5.9 | 189 | 11 | 0 | 11 | 5 | 6 | | T | | |
| | 1, member C14 | | | | | | | | | | | | | |
| 16* | Aldo-keto reductase family | gi\|13487925 | 37 | 8.5 | 120 | 10 | 0 | 10 | 7 | 3 | | T | C | |
| | 1, member C6 | | | | | | | | | | | | | |
| 17 | Aldolase 1, A isoform | gi\|53733633 | 39.3 | 9.2 | 213 | 12 | 1 | 11 | 11 | 1 | | T | | [36] |
| 18* | Aldolase 3 | gi\|60687506 | 39.4 | 6.5 | 153 | 5 | 0 | 5 | 4 | 1 | | T | M | |
| 19 | Alpha enolase (spot 4501) | gi\|58476212 | 47.1 | 6.4 | 341 | 45 | 22 | 23 | 33 | 12 | 0.24 | 0.22 | C | [33, 37-39] |
| 20 | Alpha enolase (spot 4516) | gi\|58476212 | 47.1 | 6.4 | 338 | 40 | 21 | 19 | 30 | 10 | 0.161 | 0.47 | C | [33, 37-39] |
| 21 | Alpha enolase (spot 4524) | gi\|58476212 | 47.1 | 6.4 | 338 | 42 | 22 | 20 | 31 | 11 | 0.021 | 0.53 | C | [33, 37-39] |
| 22 | Alpha enolase (spot 5510) | gi\|58476212 | 47.1 | 6.4 | 330 | 45 | 22 | 23 | 34 | 11 | 0.234 | 0.76 | C | [33, 37-39] |
| 23* | Alpha glucosidase 2 | gi\|26326711 | 106.9 | 5.6 | 269 | 5 | 0 | 5 | 0 | 5 | | T | ER, G | |
| 24 | Annexin A6 | gi\|31981302 | 75.9 | 5.3 | 267 | 5 | 0 | 5 | 0 | 5 | | T | C | [34, 35] |
| 25 | Apoa4 protein | gi\|14789706 | 45 | 5.3 | 224 | 21 | 6 | 15 | 18 | 3 | 0.011 | 2.77 | ES | [40] |
| 26 | Apoe | gi\|71060041 | 35.9 | 5.5 | 220 | 31 | 14 | 17 | 21 | 10 | 0.01 | 3.97 | ES | [34, 41] |
| 27 | Apolipoprotein A-I (spot | gi\|26345182 | 30.6 | 5.4 | 173 | 38 | 21 | 17 | 28 | 10 | 0.001 | 5.4 | S | [33, 40, 42] |
| | 2215) | | | | | | | | | | | | | |
| 28 | Apolipoprotein A-I (spot | gi\|26345182 | 30.6 | 5.4 | 170 | 30 | 18 | 12 | | | | T | S | [40, 42] |
| | 3204) | | | | | | | | | | | | | |
| 29 | Arginase 1, liver | gi\|7106255 | 34.8 | 6.6 | 306 | 38 | 23 | 15 | 28 | 10 | 0.013 | 0.32 | C | [37, 43-45] |
| 30* | Arginase type II | gi\|6753110 | 38.9 | 6.1 | 161 | 6 | 0 | 6 | 6 | 0 | | T | M | |
| 31 | Argininosuccinate | gi\|6996911 | 46.6 | 8.5 | 175 | 15 | 15 | 0 | 10 | 5 | | C | M | [22-27, 46] |
| | synthetase 1 | | | | | | | | | | | | | |
| 32 | ATP synthase, H+ | gi\|51980458 | 18.7 | 5.3 | 148 | 37 | 20 | 17 | 25 | 12 | 0.002 | 0.33 | M | [47] |
| | transporting, mitochondrial | | | | | | | | | | | | | |
| | F0 complex, subunit d (spot | | | | | | | | | | | | | |
| | 2120) | | | | | | | | | | | | | |
| 33 | ATP synthase, H+ | gi\|51980458 | 18.7 | 5.3 | 145 | 35 | 19 | 16 | 22 | 13 | | C | M | [47] |
| | transporting, mitochondrial | | | | | | | | | | | | | |
| | F0 complex, subunit d (spot | | | | | | | | | | | | | |
| | 3203) | | | | | | | | | | | | | |
| 34* | Beta 5-tubulin | gi\|18088719 | 49.7 | 4.7 | 267 | 11 | 1 | 10 | 7 | 4 | | T | Ck | |
| 35* | Branched chain ketoacid | gi\|31982494 | 50.4 | 8.4 | 237 | 26 | 18 | 8 | 20 | 6 | 0.018 | 0.5 | MM | |
| | dehydrogenase E1, alpha | | | | | | | | | | | | | |
| | polypeptide | | | | | | | | | | | | | |
| 36* | Butyryl Coenzyme A | gi\|16905127 | 64.8 | 6.6 | 119 | 4 | 4 | 0 | 0 | 4 | | C | MM | |
| | synthetase 1 | | | | | | | | | | | | | |
| 37* | Cai protein (Pdia4) | gi\|45219865 | 65 | 5.9 | 267 | 5 | 0 | 5 | 1 | 4 | | T | ER | |
| 38 | Capping protein alpha 1 | gi\|595917 | 32.8 | 5.2 | 114 | 11 | 0 | 11 | 7 | 4 | | T | Ck | [33] |
| | subunit (Capza1) | | | | | | | | | | | | | |
| 39 | Carbamoyl-phosphate | gi\|8393186 | 164.6 | 6.3 | 350 | 31 | 22 | 9 | 22 | 9 | 0.023 | 0.2 | M, C | [21, 34, 46, 48, |
| | synthetase 1, mitochondrial | | | | | | | | | | | | | 49] |
| 40 | Carboxylesterase MH1 | gi\|14331135 | 61.8 | 6.2 | 168 | 4 | 0 | 4 | 0 | 4 | | T | ER | [36, 50] |
| 41* | cDNA sequence BC021917 | gi\|21703976 | 59.7 | 6.3 | 170 | 4 | 0 | 4 | 1 | 3 | | T | | |
| | (dihydroxyacetone kinase 2 | | | | | | | | | | | | | |
| | homolog) | | | | | | | | | | | | | |
| 42 | Creatine kinase, brain | gi\|10946574 | 42.7 | 5.3 | 181 | 4 | 0 | 4 | 0 | 4 | | T | M, C | [33] |
| 43 | Cryz protein | gi\|13277837 | 35.3 | 8.2 | 84 | 5 | 5 | 0 | 5 | 0 | | C | C | [51] |
| 44 | Cu/Zn superoxide | gi\|226471 | 15.8 | 5.9 | 186 | 39 | 21 | 18 | 27 | 12 | 0.007 | 2.92 | | [33, 42] |
| | dismutase 1513495A | | | | | | | | | | | | | |
| 45 | DEMSMC malate | gi\|319837 | 36.5 | 5.9 | 138 | 31 | 17 | 14 | 19 | 12 | 0.00004 | 0.27 | C | [52] |
| | dehydrogenase, cytosolic | | | | | | | | | | | | | |
| 46* | Dhdh protein | gi\|21618806 | 36.6 | 5.7 | 209 | 26 | 17 | 9 | 16 | 10 | 0.00012 | 0.17 | | |
| 47* | Diacetyl/L-xylulose | gi\|50400594 | 25.7 | 7.8 | 228 | 5 | 5 | 0 | 0 | 5 | | C | MEM | |
| | reductase | | | | | | | | | | | | | |
| 48* | Dmgdh protein | gi\|12836171 | 97.3 | 7.6 | 184 | 8 | 8 | 0 | 5 | 3 | | C | M | |
| | (Dimethylglycine | | | | | | | | | | | | | |
| | dehydrogenase, | | | | | | | | | | | | | |
| | mitochondrial) | | | | | | | | | | | | | |
| 49* | Enoyl coenzyme A | gi\|7949037 | 36.1 | 7.4 | 162 | 26 | 11 | 15 | 21 | 5 | 0.026 | 0.49 | M, P | |
| | hydratase 1, peroxisomal | | | | | | | | | | | | | |
| 50 | Eukaryotic translation | gi\|33859482 | 95.3 | 6.3 | 208 | 6 | 0 | 6 | 3 | 3 | | T | C | [91] |
| | elongation factor 2 | | | | | | | | | | | | | |
| 51 | Eukaryotic translation | gi\|124231 | 16.8 | 5.1 | 115 | 10 | 5 | 5 | 7 | 3 | 0.106 | 3.4 | C, N | [34] |
| | initiation factor 5A (eIF-5A) | | | | | | | | | | | | | |
| 52* | Farnesyl diphosphate | gi\|19882207 | 40.6 | 5.4 | 158 | 5 | 0 | 5 | 0 | 5 | | T | C | |
| | synthetase | | | | | | | | | | | | | |
| 53* | Fatty acid binding protein 5, | gi\|6754450 | 15.1 | 6.2 | 149 | 15 | 2 | 13 | 13 | 2 | 0.09 | 27.9 | C | |
| | epidermal | | | | | | | | | | | | | |
| 54 | Fibrinogen, alpha | gi\|33563252 | 61.3 | 7 | 144 | 15 | 0 | 15 | 10 | 5 | | T | | [46, 48] |
| | polypeptide | | | | | | | | | | | | | |
| 55 | Fibrinogen, B beta | gi\|33859809 | 54.8 | 6.5 | 238 | 35 | 15 | 20 | 23 | 12 | 0.0011 | 2.41 | ES | [48] |
| | polypeptide (spot 5602) | | | | | | | | | | | | | |
| 56 | Fibrinogen, B beta | gi\|33859809 | 54.8 | 6.5 | 244 | 33 | 15 | 18 | 22 | 11 | 0.187 | 3.54 | ES | [48] |
| | polypeptide (spot 5612) | | | | | | | | | | | | | |
| 7 | Fibrinogen, gamma | gi\|18044708 | 49.4 | 5.5 | 196 | 12 | 1 | 11 | 5 | 7 | | T | ES | [43, 46] |
| | polypeptide | | | | | | | | | | | | | |
| 58 | FK506 binding protein 4 | gi\|6753882 | 51.6 | 5.5 | 154 | 4 | 0 | 4 | 1 | 3 | | T | C, N | [33, 34] |
| 59 | GDP dissociation inhibitor 2 | gi\|38197560 | 50.5 | 5.8 | 220 | 5 | 0 | 5 | 0 | 5 | | T | G, C, | [34] |
| | (GDI 2) | | | | | | | | | | | | MEM | |
| 60 | Glutathione peroxidase 1 | gi\|6680075 | 22.3 | 6.2 | 215 | 25 | 18 | 7 | 18 | 7 | 0.00002 | 0.28 | C, M | [53-55] |
| 61 | Glutathione S-transferase, | gi\|6680121 | 25.7 | 7.6 | 222 | 11 | 1 | 10 | 7 | 4 | | T | C | [21, 34] |
| | mu 2 | | | | | | | | | | | | | |
| 62 | Glycine N- | gi\|34013296 | 32.7 | 6.9 | 214 | 23 | 23 | 0 | 17 | 6 | | C | C | [21, 29, 43, 56] |
| | methyltransferase (spot | | | | | | | | | | | | | |
| | 4256) | | | | | | | | | | | | | |
| 63 | Glycine N- | gi\|34013296 | 32.7 | 6.9 | 226 | 22 | 22 | 0 | 15 | 7 | | C | C | [21, 29, 43, 56] |
| | methyltransferase (spot | | | | | | | | | | | | | |
| | 5269) | | | | | | | | | | | | | |
| 64 | Glycine N- | gi\|34013296 | 32.7 | 6.9 | 218 | 20 | 20 | 0 | 15 | 5 | | C | C | [21, 29, 43, 56] |
| | methyltransferase (spot | | | | | | | | | | | | | |
| | 9105) | | | | | | | | | | | | | |
| 65 | Glycyl-tRNA synthetase | gi\|21264024 | 81.9 | 6.2 | 180 | 5 | 0 | 5 | 0 | 5 | | T | C | [57] |
| 66 | Haao protein (3- | gi\|15277547 | 32.8 | 6 | 420 | 35 | 22 | 13 | 27 | 8 | 0.006 | 0.24 | C | [34] |
| | hydroxyanthranilate 3,4- | | | | | | | | | | | | | |
| | dioxygenase) | | | | | | | | | | | | | |
| 67 | Hal protein | gi\|35505393 | 72.2 | 5.9 | 301 | 6 | 6 | 0 | 0 | 6 | | C | C | [58] |
| 68 | Hemopexin | gi\|23956086 | 51.3 | 9 | 231 | 16 | 2 | 14 | 11 | 5 | 0.323 | 7.27 | ES | [59] |
| 69 | Heterogeneous nuclear | gi\|33667042 | 60.1 | 6.6 | 156 | 8 | 0 | 8 | 8 | 0 | | T | N | [33] |
| | ribonucleoprotein L | | | | | | | | | | | | | |
| | (hnRNPL) | | | | | | | | | | | | | |
| 70 | HSP60 (spot 2604) | gi\|1334284 | 57.9 | 5.3 | 298 | 15 | 7 | 8 | 6 | 9 | 0.008 | 0.4 | MM | [33, 34, 50] |
| 71 | HSP60 (spot 2610) | gi\|1334284 | 57.9 | 5.3 | 298 | 14 | 7 | 7 | 6 | 8 | 0.012 | 0.5 | MM | [33, 34, 50] |
| 72* | Hypothetical protein | gi\|58037115 | 22.7 | 5.8 | 125 | 29 | 15 | 14 | 21 | 8 | 0.014 | 0.39 | | |
| | LOC68347 | | | | | | | | | | | | | |
| 73* | Inosine triphosphatase | gi\|31982664 | 21.9 | 5.4 | 139 | 5 | 0 | 5 | 5 | 0 | | T | C | |
| 74 | Interleukin 1 receptor | gi\|238585 | 17.7 | 5.5 | 121 | 7 | 0 | 7 | 6 | 1 | | T | S, C | [18, 50-62] |
| | antagonist protein | | | | | | | | | | | | | |
| 75* | Interleukin 25 | gi\|18250288 | 18 | 5.9 | 89 | 13 | 6 | 7 | 9 | 4 | 0.23 | 2.1 | ES | |
| 76* | Kininogen 1 | gi\|12963497 | 47.9 | 5.7 | 217 | 21 | 8 | 13 | 19 | 2 | 0.091 | 2.8 | ES | |
| 77 | Lamin-A/C | gi\|15929761 | 74.2 | 6.6 | 199 | 12 | 0 | 12 | 10 | 2 | | T | N | [21, 33] |
| 78* | LIM and SH3 protein 1 | gi\|6754508 | 30 | 6.7 | 156 | 7 | 0 | 7 | 6 | 1 | | T | Ck | |
| 79 | Liver fructose-1,6- | gi\|6688689 | 36.9 | 6.2 | 281 | 29 | 18 | 11 | 17 | 12 | 0.013 | 0.35 | | [34, 43, 48] |
| | bisphosphatase | | | | | | | | | | | | | |
| 80* | Lysophospholipase 1 | gi\|6678760 | 24.7 | 5.9 | 134 | 25 | 13 | 12 | 20 | 5 | 0.25 | 0.25 | M | |
| 81 | Major urinary protein | gi\|1839508 | 18.7 | 4.7 | 138 | 6 | 6 | 0 | 6 | 0 | | C | S | [18, 63] |
| 82* | Major vault protein | gi\|12003287 | 96 | 5.4 | 238 | 4 | 0 | 4 | 0 | 4 | | T | C | |
| 83 | MAWD binding protein | gi\|31560132 | 32 | 5.1 | 318 | 25 | 19 | 6 | 19 | 6 | 0.065 | 0.38 | | [34, 37, 43] |
| | homolog 1 | | | | | | | | | | | | | |
| 84* | Mitochondrial acyl-CoA | gi\|40538846 | 49.6 | 7 | 123 | 6 | 6 | 0 | 1 | 5 | | C | M | |
| | thioesterase 1 | | | | | | | | | | | | | |
| 85 | NADH dehydrogenase | gi\|21312012 | 20 | 8.8 | 145 | 4 | 4 | 0 | 4 | 0 | | C | M | [48, 64] |
| | (ubiquinone) 1 alpha | | | | | | | | | | | | | |
| | subcomplex, 8 | | | | | | | | | | | | | |
| 86 | NADH dehydrogenase | gi\|26331822 | 79.7 | 5.5 | 240 | 5 | 5 | 0 | 0 | 5 | | C | M | [34] |
| | (ubiquinone) Fe—S protein 1 | | | | | | | | | | | | | |
| | (Ndufs1) | | | | | | | | | | | | | |
| 87 | NADH dehydrogenase | gi\|19526814 | 51 | 8.5 | 151 | 4 | 0 | 4 | 3 | 1 | | T | M, ES | [64] |
| | (ubiquinone) flavoprotein 1 | | | | | | | | | | | | | |
| 88 | Nit protein 2 (spot 5315) | gi\|12963555 | 30.5 | 6.2 | 347 | 34 | 18 | 16 | 24 | 10 | | C | | [33, 37] |
| 89 | Nit protein 2 (spot 6201) | gi\|12963555 | 30.5 | 6.2 | 345 | 30 | 16 | 14 | 22 | 8 | 0.295 | 0.8 | | [33, 37] |
| 90* | Nucb1 protein | gi\|49117484 | 53.4 | 5 | 184 | 8 | 0 | 8 | 8 | 0 | | T | G, C, | |
| | | | | | | | | | | | | | MEM | |
| 91 | Peroxiredoxin 6 (spot 4207) | gi\|6671549 | 24.8 | 5.7 | 282 | 40 | 21 | 19 | 30 | 10 | 0.132 | 2.3 | C, L | [37] |
| 92 | Peroxiredoxin 6 (spot 5216) | gi\|6671549 | 24.8 | 5.7 | 280 | 38 | 20 | 18 | 28 | 10 | 0.018 | 0.3 | C, L | [37] |
| 93 | Phosphatidylethanolamine | gi\|53236978 | 20.8 | 5.2 | 135 | 12 | 2 | 10 | 11 | 1 | 0.008 | 2.2 | C | [42, 43, 64] |
| | binding protein | | | | | | | | | | | | | |
| 94 | Plasminogen | gi\|200403 | 90.8 | 6 | 243 | 9 | 0 | 9 | 6 | 3 | | T | S | [49, 65-67] |
| 95* | Poly(rC) binding protein 2; | gi\|6754994 | 37.5 | 6.4 | 189 | 8 | 0 | 8 | 8 | 0 | | T | N | |
| | heterogeneous nuclear | | | | | | | | | | | | | |
| | ribonucleoprotein X | | | | | | | | | | | | | |
| 96* | PREDICTED: agmatine | gi\|20848362 | 38.3 | 8 | 108 | 24 | 16 | 8 | 19 | 5 | 0.003 | 0.1 | ES | |
| | ureohydrolase | | | | | | | | | | | | | |
| | (agmatinase) | | | | | | | | | | | | | |
| 97 | Prohibitin | gi\|54035592 | 29.8 | 5.4 | 335 | 39 | 21 | 18 | 28 | 11 | 0.018 | 0.6 | M | [33] |
| 98* | Psmd11 protein | gi\|33585718 | 46.9 | 6.1 | 258 | 4 | 0 | 4 | 0 | 4 | | T | C | |
| 99 | Pyridoxine 5′-phosphate | gi\|19527238 | 30.1 | 8.4 | 120 | 24 | 14 | 10 | 16 | 8 | 0.026 | 0.3 | | [21, 43] |
| | oxidase | | | | | | | | | | | | | |
| 100 | Pyruvate kinase 3 | gi\|31981562 | 57.8 | 7.9 | 274 | 7 | 0 | 7 | 4 | 3 | | T | M | [80] |
| 101 | Pzp protein (a2- | gi\|34785996 | 165.9 | 6.2 | 151 | 14 | 2 | 12 | 9 | 5 | | T | ES | [68] |
| | macroglobulin) | | | | | | | | | | | | | |
| 102 | Retinol binding protein 4, | gi\|33859612 | 23.2 | 5.6 | 144 | 19 | 2 | 17 | 14 | 5 | 0.129 | 12.7 | ES | [37] |
| | plasma | | | | | | | | | | | | | |
| 103* | RIKEN cDNA 1810013B01 | gi\|27753960 | 22.5 | 5.6 | 172 | 31 | 16 | 15 | 23 | 8 | 0.042 | 0.6 | N, C | |
| | (abhydrolase domain | | | | | | | | | | | | | |
| | containing 14b) | | | | | | | | | | | | | |
| 104* | RIKEN cDNA 2410004H02 | gi\|26080429 | 84.8 | 5.8 | 114 | 4 | 0 | 4 | 0 | 4 | | T | | |
| 105* | Rps12 protein | gi\|34849622 | 14.5 | 7.3 | 90 | 8 | 1 | 7 | 6 | 2 | 0.008 | 2 | C | |
| 106* | Sars1 protein | gi\|14250361 | 58.4 | 5.9 | 199 | 4 | 0 | 4 | 0 | 4 | | T | C | |
| 107 | Selenium binding protein 1 | gi\|22164798 | 52.5 | 5.9 | 336 | 31 | 19 | 12 | 21 | 10 | 0.061 | 0.5 | C, | [34] |
| | | | | | | | | | | | | | MEM, N | |
| 108 | Serine (or cysteine) | gi\|6678097 | 42.6 | 5.4 | 137 | 5 | 0 | 5 | 0 | 5 | | T | C | [33] |
| | proteinase inhibitor, clade | | | | | | | | | | | | | |
| | B, member 6a | | | | | | | | | | | | | |
| 109* | Serpinb1a protein | gi\|12834891 | 42.6 | 5.7 | 255 | 8 | 8 | 0 | 4 | 4 | | C | C | |
| 110 | Serum amyloid P- | gi\|38174334 | 26.2 | 6 | 141 | 4 | 0 | 4 | 0 | 4 | | T | ES | [21, 34, 35] |
| | component | | | | | | | | | | | | | |
| 111* | Sorcin | gi\|13385076 | 20.3 | 4.9 | 129 | 7 | 0 | 7 | 6 | 1 | | T | C, MEM | |
| 112* | T43799 proteasome protein | gi\|11265288 | 45.6 | 7.6 | 222 | 6 | 0 | 6 | 6 | 0 | | T | C, N | |
| | p45/SUG [imported] | | | | | | | | | | | | | |
| 113 | T-complex protein 1, theta | gi\|12846632 | 49.9 | 5.5 | 189 | 5 | 0 | 5 | 0 | 5 | | T | C | [21] |
| | subunit (TCP-1-theta) | | | | | | | | | | | | | |
| | (CCT-theta) | | | | | | | | | | | | | |
| 114 | Transglutaminase 2, C | gi\|6678329 | 77.1 | 5 | 240 | 6 | 0 | 6 | 3 | 3 | | T | C, MEM | [34] |
| | polypeptide | | | | | | | | | | | | | |
| 115 | Transthyretin | gi\|7305599 | 15.8 | 5.5 | 127 | 18 | 6 | 12 | 12 | 6 | 0.169 | 3 | ES | [35, 40] |
| 116 | Tumor metastatic process- | gi\|51980604 | 17.2 | 6.4 | 134 | 9 | 0 | 9 | 7 | 2 | | T | | [33, 34] |
| | associated protein NM23 | | | | | | | | | | | | | |
| 117* | Uap1I1 protein | gi\|28175154 | 56.6 | 5.2 | 168 | 4 | 0 | 4 | 0 | 4 | | T | | |
| 118 | UDP-glucose | gi\|6678499 | 54.8 | 7.4 | 307 | 14 | 0 | 14 | 13 | 1 | | T | | [57] |
| | dehydrogenase | | | | | | | | | | | | | |
| 119 | Unnamed protein product | gi\|52987 | 27.5 | 8.8 | 136 | 6 | 0 | 6 | 5 | 1 | | T | N, C | [33] |
| | (Galectin-3) | | | | | | | | | | | | | |
| 120* | v-crk sarcoma virus CT10 | gi\|56205173 | 33.8 | 5.3 | 187 | 8 | 0 | 8 | 8 | 0 | | T | C, MEM | |
| | oncogene homolog | | | | | | | | | | | | | |
| 121 | Vimentin | gi\|31982755 | 53.7 | 5 | 260 | 26 | 11 | 15 | 21 | 5 | 0.022 | 4 | Ck | [33, 50] |
| 122 | Vitamin D-binding protein | gi\|193446 | 53.1 | 5.2 | 229 | 32 | 15 | 17 | 22 | 10 | 0.175 | 1.6 | ES | [69, 70] |

TABLE 2

| | | Accession | Ratio_{HCC/control} | Ratio_{HCC/control} |
|---|---|---|---|---|
| N° | Protein | number | (serum) | (liver) |
| 1 | Alpha-fetoprotein | gi\|42542817 | 2 | up |
| | | | | (by Western |
| | | | | blot) |
| 2 | Apolipoprotein A1 | gi\|26345182 | tumor | Spot 1: 5.4 |
| | | | | Spot 2: tumor |
| 3 | Apolipoprotein E | gi\|6753102 | 2.2 | 3.9 |
| 4 | Carboxylesterase precursor | gi\|2921308 | tumor | tumor |
| 5 | Fibrinogen, alpha polypeptide | gi\|33563252 | tumor | tumor |
| 6 | Fibrinogen, beta polypeptide | gi\|33859809 | tumor | Spot 1: 2.4 |
| | | | | Spot 2: 3.5 |
| 7 | Fibrinogen, gamma | gi\|19527078 | tumor | tumor |
| | polypeptide | | | |
| 8 | Major urinary protein 1 | gi\|8569601 | 0.1 | control |
| 9 | Pzp (A2mg protein) | gi\|34785996 | Spot 1: 1.8 | tumor |
| | | | Spot 2: 3.2 | |
| 10 | Serum amyloid P-component | gi\|38174334 | 10 | tumor |

TABLE 3

| | | | Accession | Ratio | |
|---|---|---|---|---|---|
| No. | Protein name | Proteinfunction | number | T/C | Tumor association |
| 18* | Aldolase 3 | Carbohydrate metabolism | gi\|60687506 | T | occurrance in brain |
| | | | | | cancer [92] |
| 23* | Alpha glucosidase 2 | Carbohydrate metabolism | gi\|26326711 | T | |
| 9* | Alanyl-tRNA synthetase | Translation | gi\|34610207 | T | |
| 106* | Sars1 protein | Translation | gi\|14250361 | T | |
| 7* | Akr1c12 protein | Fat metabolism | gi\|15215042 | 1.9 | |
| 15* | Aldo-keto reductase family 1, | Fat metabolism | gi\|19527294 | T | Aldo-keto-reductase 1 |
| | member C14 | | | | B10 in human HCC [81] |
| 16* | Aldo-keto reductase family 1, | Fat metabolism | gi\|13487925 | T | Aldo-keto-reductase 1 |
| | member C6 | | | | B10 in human HCC [81] |
| 36* | Butyryl Coenzyme A synthetase 1 | Fat metabolism | gi\|16905127 | C | |
| 49* | Enoyl coenzyme A hydratase 1, | Fat metabolism | gi\|7949037 | 0.49 | occurrance in gastric |
| | peroxisomal | | | | carcinoma [93] |
| 52* | Farnesyl diphosphate | Fat metabolism | gi\|19882207 | T | upregulated in colon |
| | synthetase | | | | rectal cancer [71] |
| 53* | Fatty acid binding protein 5, | Fat metabolism | gi\|6754450 | 27.9 | occurrance in brain |
| | epidermal | | | | cancer [92] |
| 80* | Lysophospholipase 1 | Fat metabolism | gi\|6678760 | 0.25 | |
| 84* | Mitochondrial acyl-CoA | Fat metabolism | gi\|40538846 | C | |
| | thioesterase 1 | | | | |
| 34* | Beta 5-tubulin | Cytoskeletal proteins | gi\|18088719 | T | Elevated levels of β4b |
| | | | | | tubulin in rat HCC [82] |
| | | | | | Lung cancer [94] |
| 78* | LIM and SH3 protein 1 | This protein functions as an | gi\|6754508 | T | occurrance in breast |
| | | actin-binding protein and | | | cancer [95] |
| | | possibly in | | | |
| | | cytoskeletal organization | | | |
| | | [75). | | | |
| 105* | Rps12 protein | Ribosom component | gi\|34849622 | 2 | occurrance in breast |
| | | | | | cancer [95] |
| 98* | Psmd11 protein | Proteasom component | gi\|33585718 | T | occurrance in breast |
| | | | | | cancer [85] |
| 112* | T43799 proteasome protein | Proteasom component | gi\|11265288 | T | |
| | p45/SUG [imported] | | | | |
| 4* | 4931406C07Rik [Ester | Hydrolase | gi\|71059921 | 1.5 | |
| | hydrolase C11orf54 homolog) | | | | |
| | [spot 4342) | | | | |
| 5* | 4931406C07Rik [Ester | Hydrolase | gi\|71059921 | 1.9 | |
| | hydrolase C11orf54 homolog) | | | | |
| | [spot 4349) | | | | |
| 73* | Inosine triphosphatase | Hydrolase | gi\|31982664 | T | |
| 96* | PREDICTED: agmatine | Hydrolase | gi\|20848362 | 0.1 | Downregulation in _{“}Clear |
| | ureohydrolase [agmatinase) | | | | cell type” in renal cell |
| | | | | | carcinoma[83] |
| 103* | RIKEN cDNA 1810013B01 | Hydrolase | gi\|27753960 | 0.6 | |
| | [abhydrolase domain containing | | | | |
| | 14b) | | | | |
| 95* | Poly[rC) binding protein 2; | RNA binding Protein | gi\|6754994 | T | |
| | heterogeneous nuclear | | | | |
| | ribonucleoprotein X | | | | |
| 35* | Branched chain ketoacid | Aminoacid metabolism | gi\|31982494 | 0.5 | |
| | dehydrogenase E1, alpha | | | | |
| | polypeptide | | | | |
| 48* | Dmgdh protein [Dimethylglycine | Aminoacid metabolism | gi\|12836171 | C | |
| | dehydrogenase, mitochondrial) | | | | |
| 1* | 170 kDa glucose regulated | | gi\|7643979 | T | |
| | protein GRP170 Precurser | | | | |
| 2* | 2 hydroxyphytanoyl-CoA lyase | A peroxisomal enzyme | gi\|18204150 | 0.38 | |
| | | involved in the catabolism | | | |
| | | of phytanoic acid [76) | | | |
| 30* | Arginase type II | Arginase converts L- | gi\|6753110 | T | Upregulated in human |
| | | arginine into L-ornithine | | | pulmonary cancer [73] |
| | | and urea.[77) | | | |
| | | Arginase II is usually not | | | |
| | | expressed in liver tissue. | | | |
| 37* | Cai protein [Pdia4) | Disulfide bond isomerase | gi\|45219865 | T | |
| 46* | Dhdh protein | oxidizes trans-dihydrodiols | gi\|21618806 | 0.17 | occurrance in gastric |
| | | of aromatic hydrocarbons to | | | Cancer [96] |
| | | the corresponding catechols | | | |
| | | (99) | | | |
| 47* | Diacetyl/L-xylulose reductase | Aldo-keto reductase | gi\|50400594 | C | occurrance in prostate |
| | | | | | andenocarcinoma [93] |
| 72* | Hypothetical protein | | gi\|58037115 | 0.39 | |
| | LOC68347 | | | | |
| 75* | Interleukin 25 | Cytokine | gi\|18250288 | 2.1 | |
| 76* | Kininogen 1 | Precurser to kinin | gi\|12963497 | 2.8 | occurrance in lung |
| | | | | | adenocarcinoma [97] |
| 82* | Major vault protein | Resistance related protein | gi\|12003287 | T | occurrance in lung |
| | | | | | cancer [98] |
| 90* | Nucb 1 protein | CALNUC [nucleobindin) is | gi\|49117484 | T | occurrance in colon |
| | | an EF-hand, Ca²⁺-binding | | | carcinoma [89] |
| | | protein[79) | | | |
| 104* | RIKEN cDNA 2410004H02 | | gi\|26080429 | T | |
| 109* | Serpinb1a protein | Serinproteaseinhibitor | gi\|12834891 | C | |
| 111* | Sorcin | Ca-binding protein | gi\|13385076 | T | |
| | | associated with cardiac | | | |
| | | ryanodine receptors and L- | | | |
| | | type Ca²⁺ channels (100) | | | |
| 117* | Uap1l1 | | gi\|28175154 | T | |
| 120* | v-crk sarcoma virus CT10 | intracellular signaling | gi\|56205173 | T | Delays apoptoses in P12 |
| | oncogene homolog | cascade and in the | | | renal tumor-zellen [84] |
| | | activation of the | | | |
| | | phosphoinositide 3-kinase | | | |
| | | PI3K/AKT pathway [30] | | | |

TABLE 4

| No. | Protein | References | HCC | Other cancer | unknown |
|---|---|---|---|---|---|
| 1* | 170 kDa glucose regulated | [31, 32] | | | x |
| | protein GRP170 precursor | | | | |
| 2* | 2-hydroxyphytanoyl-CoA | | | | x |
| | lyase | | | | |
| 3 | 3-phosphoglycerate | [33] | x | Breast cancer | |
| | dehydrogenase | | | [A19] | |
| 4* | 4931406C07Rik (Ester | | | | x |
| | hydrolase C11orf54 homolog) | | | | |
| | (spot 4342) | | | | |
| 5* | 4931406C07Rik (Ester | | | | x |
| | hydrolase C11orf54 homolog) | | | | |
| | (spot 4349) | | | | |
| 6 | Acylpeptide hydrolase; N- | [34] | x | | |
| | acylaminoacyl peptide | | | | |
| | hydrolase | | | | |
| 7* | Akr1c12 protein | | | | x |
| 8 | Akr1c18 protein (aldo-keto | [21] | x | | |
| | reductase family I, member | | | | |
| | C18) | | | | |
| 9* | Alanyl-tRNA synthetase | | | | x |
| 10 | Albumin 1 (spot 3707) | [21, 35] | x | | |
| 11 | Albumin 1 (spot 3712) | [21, 35] | x | | |
| 12 | Albumin 1 (spot 4506) | [21, 35] | x | | |
| 13 | Albumin 1 (spot 4702) | [21, 35] | x | | |
| 14 | Albumin 1 (spot 5509) | [21, 35] | x | | |
| 15* | Aldo-keto reductase family I, | | | | x |
| | member C14 | | | | |
| 16* | Aldo-keto reductase family I, | | | | x |
| | member C6 | | | | |
| 17 | Aldolase I, A isoform | [36] | x | Pancreatic ductal | |
| | | | | adenocarcinoma | |
| | | | | [A1] | |
| 18* | Aldolase 3 | | | Brain cancer [A2] | x |
| 19 | Alpha enolase (spot 4501) | [33, 37-39] | x | | |
| 20 | Alpha enolase (spot 4516) | [33, 37-39] | x | | |
| 21 | Alpha enolase (spot 4524) | [33, 37-39] | x | | |
| 22 | Alpha enolase (spot 5510) | [33, 37-39] | x | | |
| 23* | Alpha glucosidase 2 | | | | x |
| 24 | Annexin A6 | [34, 35] | x | Inhibits | |
| | | | | rassignalling in | |
| | | | | breast cancer | |
| | | | | [A20] | |
| 25 | Apon4 protein | [40] | x | | |
| 26 | Apoe | [34, 41] | x | Gastric cancer | |
| | | | | [A3] | |
| | | | | Lung | |
| | | | | adenocarcinoma | |
| | | | | [A19] | |
| 27 | Apolipoprotein A-1 | [33, 40, 42] | x | Brain cancer [A2] | |
| | (spot 2215) | | | Lung | |
| | | | | adenocarcinoma | |
| | | | | [A19] | |
| | | | | Thyroid cancer | |
| | | | | [A29] | |
| 28 | Apolipoprotein A-1 | [40, 42] | x | Brain cancer [A2] | |
| | (spot 3204) | | | Lung | |
| | | | | adenocarcinoma | |
| | | | | [A19] | |
| | | | | Thyroid cancer | |
| | | | | [A29] | |
| 29 | Arginase 1, liver | [37, 43-45] | x | Gastric | |
| | | | | cancer[,A30, A31] | |
| | | | | colorectal | |
| | | | | cancer[A32, A33] | |
| | | | | Breast | |
| | | | | cancer[A34, A35] | |
| | | | | skin cancer lung | |
| | | | | cancer[A36, A37] | |
| | | | | Prostate | |
| | | | | cancer[A38] | |
| 30* | Arginase type II | | | Lung cancer | x |
| 31 | Argininosuccinate sythetase 1 | [22-27, 46] | x | Ovarian cancer | |
| | | | | [A21] | |
| 32 | ATP synthase, H+ | [47] | x | Brain cancer [A2] | |
| | transporting, mitochondrial FD | | | | |
| | complex, subunit d (spot | | | | |
| | 2120) | | | | |
| 33 | ATP synthase, H+ | [47] | x | Brain cancer [A2] | |
| | transporting, mitochondrial F0 | | | | |
| | complex, subunit d (spot | | | | |
| | 3203) | | | | |
| 34* | Beta 5-tubulin | | | Lung cancer [A5] | x |
| | | | | ↓ | |
| 35* | Branched chain ketoacid | | | | x |
| | dehydrogenase B1, alpha | | | | |
| | polypeptide | | | | |
| 36* | Butyryl Coenzyme A | | | | x |
| | synthetase I | | | | |
| 37* | Cai protein (Pdia4) | | | | x |
| 38 | Capping protein alpha 1 | [33] | x | | |
| | subunit | | | | |
| 39 | Carbamoyl-phosphate | [21, 34, 46, 48, | x | | |
| | synthetase 1, mitochondrial | 49] | | | |
| 40 | Carboxylesterase MH1 | [36, 50] | x | | |
| 41* | cDNA sequence BOD21917 | | | | x |
| | (dihydroxyacetone kinase 2 | | | | |
| | homolog) | | | | |
| 42 | Creatine kinase, brain | [33] | x | Bran cancer [A2] | |
| 43 | Cryz protein | [51] | x | | |
| 44 | Cu/Zn superoxide diamutase | [33, 42] | x | | |
| | 1513495A | | | | |
| 45 | DEMSMC malate | [52] | x | | |
| | dehydrogenase, cytosolic | | | | |
| 46* | Dhdh protein | | | Gastric Cancer | x |
| | | | | [A7] | |
| 47* | Diacetyl/L-xyulose reductase | | | Prostate | x |
| | | | | adenocarcinoma | |
| | | | | [A22] | |
| 48* | Dmgdh protein | | | | x |
| | (Dimethylglycine | | | | |
| | dehydrogenase | | | | |
| | mitochondrial) | | | | |
| 49* | Enoyl coenzyme A hydralase | | | Gastric | |
| | 1, peroxisomal | | | carcinoma [A10] | |
| 50 | Eukaryotic translation | [91] | x [A11] | | |
| | elongation factor 2 | | | | |
| 51 | Eukaryotic translation | [34] | x | Ovarian Cancer | |
| | initiation factor 5A (eIF-5A) | | | [A23] | |
| 52* | Farnesyl diphosphate | | | Colon rectal | x |
| | synthetase | | | carcinoma | |
| 53* | Fatty acid binding protein 5, | | | Brain cancer [A2] | x |
| | epidermal | | | | |
| 54 | Fibrinogen, alpha polypeptide | [46, 48] | x | | |
| 55 | Fibrinogen, B beta | [48] | x | Pancreatic ductal | |
| | polypeptide (spot 5602) | | | adenocarcinoma | |
| | | | | [A1] | |
| | | | | Thyroid cancer | |
| | | | | [A29] | |
| 56 | Fibrinogen, B beta | [48] | x | Pacreatic ductal | |
| | polypeptide (spot 5612) | | | adenocarcinoma | |
| | | | | [A1] | |
| | | | | Thyroid cancer | |
| | | | | [A29] | |
| 57 | Fibrinogen, gamma | [43, 46] | x | | |
| | polypeptide | | | | |
| 58 | FK506 binding protein 4 | [33, 34] | x | Glioma tumors | |
| | | | | [A24] | |
| 59 | GDP dissociation inhibitor 2 | [34] | x | Pancreatic | |
| | | | | carcinoma [A9] | |
| 60 | Glutathione peroxidase l | [53-55] | x | | |
| 61 | Glutathione S-transferase, mu 2 | [21, 34] | x | | |
| 62 | Glycine N-methyltransferase | [21, 29, 43, 56] | x | Cholangiocarcarcinoma | |
| | (spot 4256) | | | [A8] | |
| 63 | Glycine N-methyltransferase | [21, 29, 43, 56] | x | Cholangiocarcarcinoma | |
| | (spot 5269) | | | [A8] | |
| 64 | Glycine N-methyltransferase | [21, 29, 43, 56] | x | Cholangiocarcarcinoma | |
| | (spot 9105) | | | [A8] | |
| 65 | Glycyl-tRNA sythetase | [57] | x | | |
| 66 | Hano protein (3- | [34] | x | | |
| | hydroxyanthranilate 3,4- | | | | |
| | dioxygenase) | | | | |
| 67 | Hal protein | [58] | x | | |
| 68 | Hemopexin | [59] | x | | |
| 69 | Heterogeneous nuclear | [33] | x | Pancreatic ductal | |
| | ribonucleoprotein L | | | adenocarcinoma | |
| | | | | [A1] | |
| 70 | HSP60 (spot 2604) | [33, 34, 50] | x | Brain cancer [A2] | |
| 71 | HSP60 (spot 2610) | [33, 34, 50] | x | Brain cancer [A2] | |
| 72* | Hypothetical protein | | | | x |
| | LOC68347 | | | | |
| 73* | Inosine triphosphatase | | | | x |
| 74 | Interleukin 1 receptor | [18, 50-62] | x | | |
| | antagonist protein | | | | |
| 75* | Interleukin 25 | | | | x |
| 76* | Kininogen 1 | | | Lung | x |
| | | | | adenocarcinoma | |
| | | | | [A19] | |
| 77 | Lamin-A/C | [21, 33] | x | Colorectal Cancer | |
| | | | | [A12] | |
| 78* | LIM and SH3 protein 1 | | | Breast cancer | x |
| | | | | [A13] | |
| 79 | Liver fructose-1,6- | [34, 43, 48] | x | | |
| | bisphosphatase | | | | |
| 80* | Lysophospholipase 1 | | | | x |
| 81 | Major urinary protein | [18, 63] | x | | |
| 82* | Major vault protein | | | Lung cancer [98] | x |
| 83 | MAWD binding protein | [34, 37, 43] | x | Breast Cancer | |
| | homolog 1 | | | [A14] | |
| 84* | Mitochondrial acyl-CoA | | | | x |
| | thioesterase 1 | | | | |
| 85 | NADH dehydrogenase | [48, 64] | x | | |
| | (ubiquinone) 1 alpha | | | | |
| | subcomplex, 8 | | | | |
| 86 | NADH dehydrogenase | [34] | x | Gastric | |
| | (ubiquinone) Fe—S protein 1 | | | carcinoma [A25] | |
| | (Ndufs1) | | | | |
| 87 | NADH dehydrogenase | [64] | x | | |
| | (ubiquinone) flavoprotein 1 | | | | |
| 88 | Nit protein 2 (spot 5315) | [33, 37] | x | | |
| 89 | Nit protein 2 (spot 6201) | [33, 37] | x | | |
| 90* | Nucb1 protein | | | Colon cancer | x |
| | | | | [A41] | |
| 91 | Peroxiredoxin 6 (spot 4207) | [37] | x | Breast cancer | |
| | | | | [A15] | |
| 92 | Peroxiredoxin 6 (spot 5216) | [37] | x | Breast cancer | |
| | | | | [A15] | |
| 93 | Phosphatidylethanolamine | [42, 43, 64] | x | | |
| | binding protein | | | | |
| 94 | Plasminogen | [49, 65-67] | x | Lung | |
| | | | | adenocarcinoma | |
| | | | | [A19] | |
| 95* | Poly(rC) binding protein 2; | | | | x |
| | heterogeneous nuclear | | | | |
| | ribonucleoprotein X | | | | |
| 96* | PREDICTED: [symbol] | | | | x |
| | ureohydrolase ( [symbol] ) | | | | |
| 97 | Prohibitin | [33] | x | Gastric cancer | |
| | | | | [A27] | |
| 98* | Psmd11 protein | | | Breast cancer | x |
| | | | | [A16] | |
| 99 | Pyridoxine 5′-phosphate | [21, 43] | x | | |
| | oxidase | | | | |
| 100 | Pyruvate kinase 3 | [80] | Serum HCC | | |
| 101 | P2p protein (a2- | [68] | x | | |
| | macroglobulin) | | | | |
| 102 | Retinol binding protein 4, | [37] | x | | |
| | plasma | | | | |
| 103* | RIKEN cDNA 1810013B01 | | | | x |
| | (abhydrolase domain | | | | |
| | containing 14b) | | | | |
| 104* | RIKEN cDNA 2410004H02 | | | | x |
| 105* | Rps12 protein | | | Breast cancer | x |
| | | | | [A28] | |
| 106* | Sars1 protein | | | | x |
| 107 | Selenium binding protein 1 | [34] | x | Gastric | |
| | | | | carcinoma [A25] | |
| 108 | Serine (or cysteine) proteinase | [33] | x | | |
| | inhibitor, clade B, member 6a | | | | |
| 109* | Serpinb1a protein | | | | x |
| 110 | Serum amyloid P-component | [21, 34, 35] | x | Neuroblastoma[A29] | |
| 111* | Sorcin | | | Malignant glioma | x |
| | | | | [A17] | |
| 112* | T43799 proteasome protein | | | | x |
| | p45/SUG [imported] | | | | |
| 113 | T-complex protein 1, theta | [21] | x | | |
| | subunit (TCP-1-theta) (CCT- | | | | |
| | theta) | | | | |
| 114 | Transglutaminase 2, C | [34] | x | | |
| | polypeptide | | | | |
| 115 | Transthyretin | [35, 40] | x | Thyroid cancer | |
| | | | | [A28] | |
| 116 | Tumor metastatic process- | [33, 34] | | | x |
| | associated protein NM23 | | | | |
| 117* | Unp1l1 protein | | | | x |
| 118 | UDP-glucose dehydrogenase | [57] | x | | |
| 119 | Unnamed protein product | [33] | x | Brain cancer [A2] | |
| | (Galectin-3) | | | | |
| 120* | v-crk sarcoma virus CT10 | | | Delays apoptosis | x |
| | oncogene homolog | | | in renal tumor | |
| 121 | Vimentin | [33, 50] | x | Prostate cancer | |
| | | | | [A18] | |
| 122 | Vitamin D-binding protein | [69, 70] | x | Lung | |
| | | | | adenocarcinoma | |
| | | | | [A19] | |

[A1] Shirai, Y.; Sogawa, K.; Yamaguchi, T.; Sudo, K.; Nakagawa, A.; Sakai, Y.; Ishihara, T.; Sunaga, M.; Nazu, M.; Tomonaga, T.; Miyazaki, M.; Saisho, H.; Nomura, F. Protein profiling in pancreatic juice for detection of intraductal papillary mucinous neoplasm of the pancreas. Hepatogastroenterolgy 2008, 55(86-87), 1824-9
[A2] Khalil, A. A. Biomarker discovery: a proteomic approach for brain cancer profiling. Cancer Sci. 2007, 98(2), 201-13.
[A3] Sakashito, K.; Tanaka, F.; Zhang, X.; Mimori, K.; Kamohara, Y.; Inoua, H.; Sawada, T.; Hirakawa, K.; Mori, M. Clinical significance of ApoE expression in human gastric cancer. Oncol Rep. 2008, 20(6), 1313-9.
[A4] Graboń, W.; Mielczarek-Puta, M.; Chrzenowska, A.; Barańczyk-Kuźma, A. l-Arginine as a factor increasing arginase significance in diagnosis of primary and metastatic colorectal cancer. Clin. Biochem. 2008 Dec. 11. [Epub ahead of print]
[A5] [symbol] , V.; Hiser, L.; Smith, H.; Frankfurter, A.; Spano, A.; Corrola, J. J.; Lobert, S. Beta-tubulin isotype classes II and V expression patterns in nonsmall cell lung carcinomas. Cell Motif Cytoskeleton. 2006, 65(8), 675-85.
[A6] Francis, G.; Mitchell, S. D.; Moss, S. E.; Hanby, A. M.; Marshall, J. F.; Hart, I. R. Identification by differential display of annexin-VI, a gene differentially expressed during melanoma progression. Cancer Res. 1996, 56(17), 3855-8.
[A7] Chang, H. C.; Chen, Y. L.; Chan, C. P.; Yeh, K. T.; Kuro, S. J.; Ko, C. J.; Fang, H. Y.; Overexpression of Dihydrodiol Dehydrogenase as a Prognostic Marker in Resected Gastric Cancer Patients. Dig Dis Sci. 2008. [Epub ahead of print]
[A8] Huang, Y. C.; Chen, M.; Shyr, Y. M.; Su, C. H.; Chen C. K.; Li, A. F.; Ho, D. M.; Chen; Y. M. Glycine N-methyltransferase is a favorable prognostic marker for human cholangiocarcinoma. J Gastroenterol Hepatol. 2008, 23(9), 1384-9. Epub 2008 Jul. 8.
[A9] Sun, Z. L.; Zhu, Y.; Wang, F. O.; Chen, R.; Peng, T.; Fan, Z N.; Xu, Z. K.; Miao, Y. Serum proteomic-based analysis of pancreatic carcinoma for the identification of potential cancer biomarkers. Biochim Biophys Acta. 2007, 1774(6), 764-71. Epub 2007 Apr. 5.
[A10] Kim, H. K.; Park, W S.; Kang, S. H.; Warda, M.; Kim, N.; Ko, J. H.; Prince, Ael-B.; Han, J. Mitochondrial alterations in human gastric carcinoma cell line. Am J Physiol Cell Physiol. 2007, 293(2), C761-71. Epub 2007 May 30.
[A11] Li, L.; Chen S. H.; Yu, C. H.; Li, Y. M.; Wang, S. O. Identification of hepatocellular-carcinoma-associated antigens and autoantibodies by serological proteome analysis combined with protein microarray. J Proteome Res. 2008, 7(2), 611-20.
[A12] Willis, N. D.; Cox, T. R.; Rahman-Casañs, S. F.; Smits, K.; Przyborski, S. A.; van den Brandt, P.; van Engeland, M.; Weijenberg, M.; Wilson, R. G.; de Bruïne, A.; Hutchison, C. J., Lamin A/C is a risk biomarker in colorectal cancer. PLoS ONE. 2008 Aug. 20; 3(8);
[A13] Grunewald, T. G.; Kammerer, U.; Kapp, M.; Eck, M.; Dietl, J.; Butt, E.; Honig, A.; Nuclear localization and cytosolic overexpression of LASP-1 correlates with tumor size and nodal-positivity of human breast carcinoma. BMC Cancer. 2007; 7, 198
[A14] Matsuda, S.; Katsumata, R.; Okuda, T.; Yamamoto, T.; Miyazaki, K.; Senga, T.; Machida, K.; Thant, A. A.; Nakataugawa, S.; Hamaguchi, M. Molecular cloning and characterization of human MAWD, a novel protein containing WD-40 repeats frequently overexpressed in breast cancer. Cancer Res. 2000, 60(1), 13-7.
[A15] Chang X Z, Li D Q, Hou Y F, Wu J, Lu J S, Di G H, Jin W, Qu Z L, Shen Z Z, Shao Z M. Identification of the functional role of peroxiredoxin 6 in the progression of breast cancer. Breast Cancer Res. 2007; 9(6): R76.
[A16] Deng, S.; Zhou, H.; Xiong, R.; Lu, Y.; Yan, D.; Xing, T.; Dong, L.; Tang, E.; Yang, H. Over-expression of genes and proteins of ubiquitin specific peptidases (USPs) and proteasome subunits (PSs) in breast cancer tissue observed by the methods of RFDD-PCR and proteomics Breast Cancer Res Treat. 2007, 104(1), 21-30
[A17] Yokota, T.; Kuono, J.; Adachi, K.; Takahashi, H.; Teramoto, A.; Matsumoto, K.; Sugisaki, Y.; Onda, M.; Tsunoda, T. Identification of histological markers for malignant glioma by genome- wide expression analysis: dynein, alpha-PIX and sorcin. Acta Neuropathol. 2006, 111(1) 29-38
[A18] Wu, M.; Bal, X.; Xu, G.; Wei, J.; Zhu, T.; Zhang, Y.; Li, Q.; Liu, P.; Song, A.; Zhao, L.; Gang, C.; Han, Z.; Wang, S.; Zhou, J.; Lu, Y.; Ma, D Proteome analysis of human androgen-independent prostate cancer celllines: variable metastatic potentials correlated with vimentin expression Proteomics. 2007, 7(12), 1973-83.
[A19] Al-Dhaheri, M. H.; Shah, Y. M.; Basrur, V.; Pind, S.; Rowan, B. G. Identification of novel proteins induced by estradiol, 4-hydroxytamoxifen and acolbifene in T47D breast cancer cells. Steroids. 2006, 71(11-12), 966-78.
[A20] de Muga, S. V.; Timpson, P.; Cubells, L.; Evans, R.; Hayes, T. E.; Rentero, C.; Hegemann, A.; [symbol] , M.; Leschner, J.; Pol, A.; Tabar, F.; Daly, R J.; Enrich, C.; Grewal T. Annexin A6 inhibits Ras signalling in breast cancer cells. Oncogene. 2008 Oct. 13. [Epub ahead of print]
[A21] Szlosarek, P. W.; Grimshaw, M. J.; Wilbanks, G. D.; Hagemann, T.; Wilson, J. L.; Burke, F.; Stamp, G.; Balkwill, F. R. Aberrant regulation of argininosuccinate sythetase by TNF-alpha in human epithelial ovarian cancer. Int J Cancer. 2007, 121(1), 8-11.
[A22] Cho-Vega, J. H.; Tsavachidis, S.; Do. K. A.; Nakagawa, J.; Medelinos, L. J.; McDonnell, T. J. Dicarbonyl/L-xylulose reductase: a potential biomarker identified by laser-capture microdissection-micro serial analysis of gene expression of human prostate adenocarcinoma. Cancer Epidermiol Biomarkers Prev. 2007, 18(12) 2615-22.
[A23] Guan X. Y.; Fung, J. M.; Ma, N. F.; Lau, S. H.; Tal, L. S.; Xie, D.; Zhang, Y.; Hu, L.; Wu, Q. L.; Fang, Y.; Sham J. S. Oncogenic role of eIF-5A2 in the development of ovarian cancer. Cancer Res. 2004, 64(12) 4197-200
[A24] Jiang, W.; Cazacu S.; Xiang, C.; Zenklusen, J. C.; Fine, H. A.; Berens M.; Armstrong B.; Brodie, C.; Mikketsen, T. FK506 binding protein mediates glioma cell growth and sensitivity to rapamycin treatment by regulating NK-kappaB signalling pathway. Neoplasia. 2008, 10(3), 235-43.
[A25] Zeng, X.; Liao, A. J.; Tang, H. L.; Yi, L.; Xie, N.; Su, Q. Screening human gastric carcinoma-associated antigens by serologic proteome analysis Al Zheng. 2007 October; 26(10): 1080-4. Chinese.
[A26] Kang, X.; Zhang, L.; Sun, J.; Ni, Z.; Ma, Y.; Chen X.; Sheng, X.; Chen, T. Prohibitin: a potential biomarker for tissue-based detection of gastric cancer. J Gastroenterol. 2008, 43(8), 618-25.
[A27] Deng, S. S.; Xing, T, Y.; Zhou, H. Y.; Xiong, R. H.; Lu, Y. G.; Wen B.; Liu, S. O.; Yang, H. J. Comparative proteome analysis of breast cancer and adjacent normal breast tissuesin human. Genomics Proteomics Bioinformatics. 2006 August: 4(3) 165-72.
[A28] Sandoval, J A.; Turner, K. E.; Hoelz, D. J.; Rescoria, F. J.; Hickey, R. J.; Malkas, L. H. Serum protein profiling to identify high-risk neuroblastoma: proclinical relevance of blood-based biomarkers. J Surg Res. 2007, 142(2), 263-74.
[A29] [symbol] , L.; Iacconi, P.; Ciregia, F.; Giannaccini, G.; Donatini, G. L.; [symbol] F.; Miccoli, P.; Pinchers, A.; Lucacchini, A. Fine-needle aspiration of thyroid nodules: proteomic analysis to identify cancer biomarkers. J Proteome Res. 2008, 7(9) 4079-88.
[A30] Wu, C. W.; Chi, C. W.; Lin, E. C.; Lui, W. Y.; P'eng, F. K.; Wang, S. R. Serum arginase level in patients with gastric cancer. J Clin. Gastroenterol. 1994, 18(1), 84-5
[A31] Wu, C. W.; Chung, W. W.; Chi, C. W.; Kao, H. L.; Lui, W. Y.; P'eng, F. K.; Wang, S. R. Immunohistochemical study of arginase in cancer of the stomach. Virchows Arch. 1996, 428(6), 325-31.
[A32] Leu, S. Y.; Wang, S. R. Clinical significance of arginase in colorectal cancer. Cancer. 1992, 70(4), 733-6.
[A33] del Ara, R. M.; González-Polo, R. A.; Caro, A.; del Arno, E.; Palomo, L.; Hernández, E.; Sotor, G.; Fuentes, J. M.; Diagnostic performance of arginase activity in colorectal cancer. Clin Exp Med. 2002, 2(1), 53-7.
[A34] Porembska, Z.; Luboinski, G.; Chrzanowaka, A.; Mielczarek, M.; Magnuska, J.; Barańczyk-Kúzma, A. Arginase in patients with breast cancer. Clin Chim Acta. 2003, 328(1-2), 105-11.
[A35] Gökmen, S. S.; Aygit, A. C., Ayhan, M. S.; Yorulmaz, F.; [symbol] , S.; Significance of arginase and ornithine in malignant tumors of the human skin. J Lab Clin Med. 2001, 137(5), 340-4.
[A36]Súer Gökmen, S.; [symbol] , Y.; Cakir, E.; Yorulmaz, F.; [symbol] , S.; Arginase and ornithine, as markers in human non-small cell lung carcimona. Cancer Biochem Biophys. 1999, 17(1-2), 125-31.
[A37] Yang, H.; Li, G.; Huang, S.; Qu, S. [Study of arginase activity in alveoloar macrophages from patients with lung cancer] [Article in Chinese]. Hunan Yi Ke Da Xue Xue Bao. 1997; 22(1) 84-6 (bin mir nicht sicher ob ich einen Chinesischen Artikel [symbol] kann. Hab die info aus dem englishen Abstract)
[A38] Keskinege, A.; [symbol] , S.; Yilmaz, E. Possible implications of arginase and diamine oxidase in prostatic carcinoma. Cancer Detect Prev. 2001, 25(1), 76-9.
[A39] Lin, Y. H.; Park, Z, Y.; Lin, D.; [symbol] , A. A.; Rio, M. C.; Yates, J. R. 3rd; Klemke, R. L. Regulation of cell migration and survival by focal adhesion targeting of Lasp-1. J Cell Biol. 2004, 185(3), 421-32.
[A40] Zhou, Y.; Xu, Y.; Tan, Y.; Qi, J.; Xiao, Y.; Yang, C.; Zhu, Z.; Xiong, D. Sorcin, an important gene associated with multidrug-resistance in human leukemia cells. Leuk Res. 2006, 30(4), 469-76.
[A41] Chen, Y.; Lin, P.; Qiu, S.; Peng, X. X.; Lool, K.; Farquhar, M. G.; Zhang, J. Y. Autoantibodies to Ca2+ binding protein [symbol] is a potential marker in colon cancer detection. Int J Oncol. 2007, 30(5), 1137-44. [symbol]
indicates data missing or illegible when filed

1. Protein regulated by excessive EGFR signalling in the liver for use as biomarker in the diagnosis, prognosis and/or monitoring of treatment, preferably in the early stage, of diseases, including liver cell dysplasia or hepatocellular carcinoma (HCC), wherein the protein is selected from a first group consisting of
Arginase type II, 4931406C07Rik (Ester hydrolase C11orf54 homolog), Akr1c12 protein, Alanyl-tRNA synthetase, Aldo-keto reductase family 1 member C14, Aldo-keto reductase family 1 member C6, Aldolase 3, Alpha glucosidase 2, Beta 5-tubulin, Cai protein (Pdia4), cDNA sequence BC021917 (dihydroxyacetone kinase 2 homolog), Farnesyl diphosphate synthetase, Fatty acid binding protein 5 epidermal, Inosine triphosphatase, Interleukin 25, Kininogen 1, LIM and SH3 protein 1, Major vault protein, Nucb1 protein, Poly(rC) binding protein 2; heterogeneous nuclear ribonucleoprotein X, Psmd11 protein, RIKEN cDNA 2410004H02, Rps12 protein, Sars1 protein, Sorcin, T43799 proteasome protein p45/SUG [imported], Uap1l1 protein, v-crk sarcoma virus CT10 oncogene homolog, 170 kDa glucose regulated protein GRP170 precursor,
or from a second group consisting of
2-hydroxyphytanoyl-CoA lyase, Branched chain ketoacid dehydrogenase E1 alpha polypeptide, Butyryl Coenzyme A synthetase 1, Dhdh protein, Diacetyl/L-xylulose reductase, Dmgdh protein (Dimethylglycine dehydrogenase, mitochondrial), Enoyl coenzyme A hydratase 1 peroxisomal, Hypothetical protein LOC68347, Lysophospholipase 1, Mitochondrial acyl-CoA thioesterase 1, PREDICTED: agmatine ureohydrolase (agmatinase), RIKEN cDNA 1810013B01 (abhydrolase domain containing 14b), Serpinb1a protein.

2. Protein according to claim 1 for use as biomarker in the diagnosis, prognosis and/or treatment monitoring of dysplasia or cancer, in particular bladder, breast, cervical, colorectal, endometrial, gastric, head and neck, ovarian and oesophageal dysplasia or cancer, wherein the protein is selected from a first group consisting of
4931406C07Rik (Ester hydrolase C11orf54 homology, Akr1c12 protein, Alanyl-tRNA synthetase, Aldo-keto reductase family 1 member C14, Aldo-keto reductase family 1 member C6, Alpha glucosidase 2, Inosine triphosphatase, Interleukin 25, Poly(rC) binding protein 2; heterogeneous nuclear ribonucleoprotein X, RIKEN cDNA 2410004H02, T43799 proteasome protein p45/SUG [imported], Uap1l1 protein, 170 kDa glucose regulated protein GRP170 precursor,
or from a second group consisting of
2-hydroxyphytanoyl-CoA lyase, Branched chain ketoacid dehydrogenase E1 alpha polypeptide, Butyryl Coenzyme A synthetase 1, Dmgdh protein (Dimethylglycine dehydrogenase, mitochondrial), Hypothetical protein LOC68347, Lysophospholipase 1, Mitochondrial acyl-CoA thioesterase 1, PREDICTED: agmatine ureohydrolase (agmatinase), RIKEN cDNA 1810013B01 (abhydrolase domain containing 14b), Serpinb1a protein.

3. Protein regulated by excessive EGFR signalling in the liver for use as serum marker in the diagnosis, prognosis and/or treatment monitoring of liver cell dysplasia or hepatocellular carcinoma (HCC) wherein the protein is selected from a first group consisting of Apolipoprotein A1, Apolipoprotein E, Carboxylesterase precursor, Fibrinogen-alpha polypeptide, Fibrinogen-beta polypeptide, Fibrinogen-gamma polypeptide, Pzp (A2mg protein), Serum amyloid P-component
or from a second group consisting of
Major urinary protein 1.

4-21. (canceled)
